(19) United States
(12) Patent Application Publication
(10) Pub. No.: US 2025/0090749 A1
(43) Pub. Date: Mar. 20, 2025

(54) MEDICAL FLUID CONTROL DEVICE

(71) Applicant: Silberspross GmbH, Zürich (CH)

(72) Inventors: Nathanael WEISS, Zürich (CH); Maurice GRUNDER, Zürich (CH); Markus WEISS, Filzbach (CH)

(73) Assignee: Silberspross GmbH, Zürich (CH)

(21) Appl. No.: 18/730,864

(22) PCT Filed: Jan. 17, 2023
(86) PCT No.: PCT/CH2023/050001

§ 371 Date: Jul. 22, 2024

(30) Foreign Application Priority Data
Jan. 26, 2022 (CH) CH000078/2022

(51) Int. Cl. A61M 5/168

(52) U.S. Cl. CPC A61M 5/16804

(57) ABSTRACT

A medical fluid control device has a base body and an actuator body. The base body and the actuator body have, respectively, a sliding and sealing surface facing each other and the actuator body is fastened movably to the base body. The actuator body has at least one actuator fluid channel which connects an actuator attachment nozzle directly to an actuator opening in the sliding and sealing surface of the actuator body. The base body has independent first and second base fluid channels, each having a base opening in the sliding and sealing surface of the base body and free of a branching in the base body. The actuator body is movable such that the actuator fluid channel can be brought into a closed or open position in connection with the first or second base fluid channel.

### FIELD OF THE INVENTION

The invention relates to a medical fluid control device, in particular a device for the uninterrupted exchange of medical infusion systems on an infusion line.

### BACKGROUND

The precise and continuous intravenous administration of potent, short-acting circulatory drugs (example: adrenaline/noradrenaline) to stabilize the circulation in patients in shock, with cardiac insufficiency or during operations or anaesthesia is nowadays generally carried out using syringe pumps.

In critically ill patients, especially neonates, infants and children, low flow rates, i.e. 0.1-7.0 ml/h, and correspondingly highly concentrated solutions are used to avoid overloading the patients with fluid, as they often require several infusions at once in addition to fluid supply for nutrition (Kim U R et al, 2017 [1]). The administration of high-concentration, short-acting medications using low flow rates carries the risk that the smallest flow fluctuations can sometimes lead to serious effects on the patient.

At low flow rates, the so-called start-up, i.e. reaching the preselected constant flow rate after starting the syringe pump, is delayed. This is due to gaps in the gearbox, mechanical play between the syringe and its mountings in the syringe pump housing and slider as well as the compressibility and elasticity (compliance) of the slider, syringe and syringe plunger tip. Start-up times of more than one hour from pressing the start button of the syringe pump to reaching 95% of the continuous flow rate at 1 ml/h preselected flow rate have been reported when using standard 50 ml infusion syringes (Neff T et al., 2001 [2]). If one takes into account that reaching a continuous plasma concentration of noradrenaline or adrenaline requires additional time from the time a continuous flow rate is reached, this takes a further 16.5 minutes at a flow rate of 1 ml/h under the best conditions when administering adrenaline 0.1 mcg/kg/min to a 3 kg newborn (Baeckert M et al., 2020 [3]). Once the flow rate has settled, the syringe pump generally infuses the medication reliably and continuously, provided that the syringe pump in its vertical position and the infusion system leading to the patient are not manipulated.

However, changing a syringe pump infusion or infusion syringe with highly concentrated, short-acting circulatory drugs at low flow rates of less than 5 ml/h that is soon to be empty is problematic.

If the empty infusion syringe is unclamped from the pump, replaced with a full syringe at the infusion line or three-way stopcock and then reinserted into the syringe pump, it can take from 10-15 minutes to an hour for the newly aligned and restarted syringe pump to resume continuous delivery at the previous run rate and a further few minutes for the medication to return to the previous plasma concentration.

In addition to the delayed start-up of the newly connected syringe pump, the pressure differences between the infusion line to the patient and the newly connected infusion line of the full syringe also contribute to flow irregularities when the syringe pump is changed. Depending on the pressure gradient, a forward infusion bolus (overdosing) or a backflow of infusion fluid into the newly connected infusion syringe pump (underdosing) occurs when the three-way stopcock is opened (Russotto V et al., 2020 [4]). The drop in the plasma concentration of the circulatory drug, such as adrenaline or noradrenaline, associated with backflow and start-up delay can lead to a life-threatening circulatory collapse, if not a circulatory collapse requiring resuscitation. Conversely, an infusion bolus of adrenaline or noradrenaline can lead to a life-threatening hypertensive circulatory situation. The pressure in the infusion line is mainly determined by the central venous pressure, the number and delivery rates of other infusions flowing through at the same time, resistance elements such as air filters, non-return valves, connectors, three-way stopcocks and the diameter and patency of the catheter lumen. The pressure in the newly attached infusion is primarily determined by the position of the syringe pump and the amount of fluid that is compressed when the infusion is connected to the three-way stopcock (Elli S et al., 2020 [5]). If so-called needle-free connectors (NFC) are used to connect the infusion line to the three-way stopcock, depending on the properties of the NFCs, a forward infusion bolus towards the patient or a backflow of infusion fluid into the newly connected syringe may also occur during connection (Elli S et al., 2020 [5]). The problems of a forward infusion bolus or backflow of fluid when connecting a new infusion or opening the three-way stopcock are largely unknown to doctors, nursing staff and the medical technology industry.

Furthermore, when changing syringe pumps with two syringe pumps connected to two different infusion ports of a three-way stopcock multiple combination, there is the problem that the medication from the newly connected infusion pump is not delivered again at the same point where the old, stopped syringe pump delivered the medication into the infusion line (multi-infusion problem). Accordingly, the dead space between the two infusion ports results in a delayed doubling or a gap in drug delivery into the bloodstream (Moss R et al., 2009 [6]).

To date, efforts to prevent flow irregularities when changing syringe pumps have focused exclusively on measures to accelerate start-up, keep the time required for the change as short as possible and compensate for the reduced initial delivery rate. There are no measures or strategies for preventing or compensating for pressure differences between the infusion line and the newly connected syringe pump in the literature, nor are they offered by the manufacturers. In order to prevent or minimize flow fluctuations when changing syringe pumps, various basic measures for accelerating the start-up and various strategies for changing syringe pumps have become established in daily practice.

As a basic measure to shorten the start-up time, a liquid bolus (approx. 1.0 ml) is always triggered manually from the newly aligned syringe pump through the infusion line into the air or into a swab using the so-called bolus button before connecting the infusion line in order to bring the pump gearbox and the syringe into position in the pump holder (Neff T et al., 2001 [2]; Baeckert M et al., 2020 [3]). This so-called bolus priming is suitable for the 2-pump change techniques and is performed on the newly prepared pump as long as it is not yet connected to the three-way stopcock.

As a further measure to shorten the start-up time, some modern syringe pumps have a so-called fast-start mode in which, for example, a mini-bolus of 0.1 ml of liquid is delivered from the syringe into the connected infusion system when the start button is activated in order to bring the pump gearbox and syringe into position in the syringe pump to overcome the compressibility and distensibility of the system and build up the initial delivery pressure for the subsequent flow. However, studies have shown that the delivery of a fluid bolus of 1 ml into the air before connecting the infusion is more effective and possibly also safer in terms of start-up than an uncontrolled delivery of a fluid bolus of 0.1 ml from the syringe pump already connected to the infusion system, i.e. to the patient (Baeckert M et al., 2020 [3]). The fast-start mode is mainly used for 1-pump exchange techniques if the newly prepared syringe is already connected to the three-way stopcock before insertion into the pump and bolus priming is no longer possible.

With the 1-pump exchange strategy, the syringe is exchanged as quickly as possible at the proximal infusion line or the infusion line with syringe at the three-way stopcock and inserted into the initial pump and then restarted.

In the 2-pump exchange strategy, the infusion syringe with the infusion line is aligned well in advance and clamped in a second syringe pump. There are then different strategies for exchanging the newly aligned syringe pump with infusion line for the old, soon to be emptied, still running syringe pump unit in order to minimize the interruption of intravenous drug delivery to the patient:

A) The quick exchange technique involves the newly directed syringe pump delivering the fluid from the end of the infusion line into a sterile swab over a certain lead time, for example 15 to 30 minutes. The delivery rate should then have reached the continuous target level. Using a quick exchange (QC) maneuver, the end of the infusion line near the patient of the soon-to-be-empty syringe pump is exchanged as quickly as possible with the end of the infusion line near the patient of the newly directed infusion at the three-way stopcock. The rapid replacement of two infusion lines on a three-way stopcock means uncontrolled rapid manipulation of infusion parts on a three-way stopcock or intravenous catheter that must be kept sterile. The open three-way stopcock or the open infusion line, if not closed otherwise, can lead to a retrograde backflow of fluid (medication) and/or blood. This in turn leads to a delay in the delivery of medication to the patient when it is restarted and possibly to thrombosis (blockage of the catheter by coagulated blood) of the catheter lumen leading to the patient. Furthermore, the area around the three-way stopcock becomes wet, which is disadvantageous from a hygienic point of view. When the infusion lines are changed quickly at the three-way stopcock, air can very easily become trapped in the fluid column. Air in the fluid column in the infusion system leads to a compressible zone (=delay in distal blood flow), to a subsequent gap (air column in the infusion line) in the drug delivery and to an air embolism, with the risk of a paradoxical arterial cerebral air embolism.

B) There are other manual and automated switching procedures with two pumps. The new syringe pump is connected to the infusion line and the three-way stopcock for the new infusion is opened. In the manual exchange procedure, the new, full syringe pump is then started and the old, almost empty syringe pump is stopped and the three-way stopcock for the old, almost empty pump is closed. With automated changeover procedures, the pumps are started and stopped automatically with a special fast-start algorithm if necessary. Pump systems with automated procedures are only occasionally offered by manufacturers (Cour M et al., 2013 [7]).

C) The double-pump technique is a procedure in which the new, full infusion syringe pump is connected to the infusion to the patient and started in parallel with the old, almost empty syringe pump infusion that is still running. The flow rate of the new, full infusion syringe pump is increased manually by the nursing staff on the basis of the hemodynamic measurements (blood pressure, pulse) and the institute's own protocols, and that of the old, almost empty infusion syringe pump is reduced to zero or the old, almost empty syringe pump is then stopped and disconnected. This procedure is very labour-intensive and often does not work without hemodynamic fluctuations. This procedure only works well if the staff are familiar and experienced with it.

The publications «Delivery interaction between co-infused medications: an in vitro modeling study of microinfusion» by Tsao A C (Tsao A C et al., 2013 [8]) and «Novel Pump Control Technology Accelerates Drug Delivery Onset in a Model of Pediatric Drug Infusion» by Parker M J et al. (Parker M J et al., 2017 [9]) show a 2-way three-way stopcock combination (venting manifold system) that allows a new full syringe pump to be connected to a carrier infusion, which drains the infusion fluid into a drainage system (vent) during the startup of the syringe pump until steady-state flow conditions are reached. When the desired continuous flow conditions are reached, the Vent three-way stopcock is turned and the drug from the newly connected syringe pump is delivered into the carrier infusion (and the old infusion can be stopped and disconnected if necessary). This arrangement avoids the hasty, rapid and uncontrolled switching of two syringe pump lines. Optionally, the old infusion can be left in place and operated (before it is disconnected) until it has been shown that the patient or their circulation remains stable.

One disadvantage of this arrangement (venting manifold system) is that the connection (dead spaces) to the carrier three-way stopcock must be pre-filled before the vent three-way stopcock is attached in order to avoid delays in the application of medication.

Dead spaces in three-way stopcock systems are always a source of risk for complications in infusion therapy. Without venting, i.e. manual flushing or pre-filling of the ports of the three-way stopcock, there is air in the cone of the connection port and possibly in the rotating part of the three-way stopcock. If an infusion is connected to the non-vented or non-prefilled three-way stopcock connection port in this way, 0.1 ml of air is first administered into the carrier infusion before the medication is delivered from the syringe pump line into the transporting carrier infusion. In addition to the risk of an air embolism, this also means a further (in addition to start-up) delay in drug delivery into the carrier infusion system or to the patient (0.1 ml at a flow rate of 1 ml/h corresponds to an additional six minute delay and additional time lost for the pressure build-up to compress the air). If the connection port of the three-way stopcock is retrogradely flushed with NaCl 0.9% or the carrier infusion or flushed out locally from the outside, the risk of air embolism is eliminated, but the delay in drug delivery still exists, as 0.1 ml of NaCl 0.9% must first be infused from the connection port of the three-way stopcock into the carrier infusion before the drug reaches the carrier infusion. Filling the connection port of three-way stopcock systems means a moist environment, unnecessary manipulation of the connection port, which favours catheter infections. The patient also does not receive 0.1 ml of the infused medication and the medication may not be effective enough, which is particularly important in pediatrics.

If the 0.1 ml residual drug in the three-way stopcock connection port is rinsed or washed out anterograde by means of an NaCl 0.9% rinse injection into the patient, there is an additional risk of fluid overload, particularly in small patients with repetitive drug administration or repeated rinsing.

If the residual drug in the three-way stopcock connection port is not rinsed out retrogradely, anterograde or locally, 0.1 ml of residual drug remains in the connection port, which is flushed into the patient during a later injection (undesirable late effect) or the newly injected drug reacts with the old residual drug and physical interactions such as precipitation occur, which can lead to blockage of the three-way stopcock due to precipitation or petrification.

There has therefore long been an urgent need for a device for the uninterrupted change of medical infusion systems on an infusion line, as well as infusion connection devices without dead space, which has still not been satisfied.

Conventional three-way stopcocks in intravenous infusion therapy as well as in arterial pressure line systems, in medical devices (heart-lung machines, dialysis machines, haemofiltration devices, ECMO devices (extracorporeal membrane oxygenation), contrast medium injectors, etc.) have the above-mentioned disadvantage when injecting fluids or aspirating fluids/blood that there is a dead space in the cone of the attachment port and in the rotating part of the three-way stopcock, which has the following disadvantages and dangers/risks (Hadaway L, 2018 [10]):
1) physical interactions of drugs with precipitation up to adhesion and malfunction of the three-way stopcock;
2) residual drugs that are washed in during the next injection and have unwanted effects (muscle relaxation, respiratory depression);
3) air in the cone with the risk of air embolism;
4) residual blood in the cone after aspiration with the risk of a wet environment due to flushing efforts using NaCl 0.9% or infections in the remaining and/or dried residual blood;
5) Inaccurate dosing of medication, especially with small volumes, as well as different handling to control the dead space during an injection (Muffly M K et al, 2017 [11]);
6) The flushing of medication/fluids/blood products from the cone of the injection port and from the dead space of the three-way stopcock rotating part into the catheter and thus into the patient can lead to fluid exposure if performed several times, especially in small patients.

Solutions to the problem of air in the cone of the rotary part of a three-way stopcock have been described in U.S. Pat. No. 7,530,546 (so-called needle-free connector) and US2013060205 or WO2017150125 (cone-flowing special three-way stopcock). In particular, the combination with an NFC (Marvelous™ Stopcock (MRVLS)) is recommended by one manufacturer in order to avoid the problem of dead space of three-way stopcocks in infusion therapy (ELCAM Medical—https://www.elcam-medical.com).

The design of a three-way stopcock with a rotary insert for changing the direction/blocking the infusion flow also has the further disadvantage that the infusion/injection resistances are increased with each installed three-way stopcock, which is particularly disadvantageous for massive infusions/transfusions (Yamaguchi K et al., 2021 [12]). The design of the three-way stopcock also allows unintentional obstruction of the infusion line due to accidentally crossed or incorrectly positioned three-way stopcocks. For example, not only one infusion can be blocked, but an entire upstream group of infusion lines can be blocked. Due to the extensibility of the multiple infusion system and the sometimes preset high alarm pressure values at which the occlusion alarm of the pump or pumps is triggered, the sounding of the occlusion alarm can be considerably delayed (minutes to hours), which makes it difficult to recognize and eliminate the blockage promptly and can lead to a stop of the intravenous drug supply to the patient and thus to a critical drop in the drug plasma concentration (Kim D W et al., 1999 [13]). If the occlusion is recognized and corrected without first relieving the built-up pressure in the infusion system by means of a three-way stopcock or by disconnecting the infusion line and draining it to the outside or against atmospheric pressure, the pressurized drug volume empties into the patient within seconds, sometimes with dramatic consequences (Kawakami H. et al., 2013 [14]).

EP3421076 describes a medical valve system with a valve housing and at least two inlets, each of which is connected in a fluid-conducting manner to an outlet arranged on an inner circumferential surface of the housing. A valve body with an outer circumferential body surface is arranged in the valve housing so as to be rotatable about an axis of rotation. An outlet is arranged on the outer circumferential surface of the body. The valve body also comprises a valve outlet, which can optionally be fluidically connected to one of the inlets. The problem with the dead volume has not been solved.

EP2937135, EP1627658, U.S. Pat. No. 6,457,488 and EP2195075 describe a valve with a housing which defines a passage line and a valve body with an inlet. In a first position, the through line is open. In a second position, the inlet is only connected to the outlet of the through line. The problem with the dead volume has not been solved.

US20070068587 describes a valve with a cylindrical body which comprises two through lines and a collar with an access line arranged around the cylindrical body. The collar can be fluidically connected to the first passage line in a first position and to the second passage line in a second position. The problem with the dead volume has not been solved because, depending on the position of the collar, there is a dead leg connected to the through line. Accordingly, the valve has dead spaces or dead leg that cannot be flushed.

### NON-PATENT LITERATURE

[1] Kim U R et al. Drug Infusion Systems: Technologies, Performance, and Pitfalls. Anesth Analg. 2017 May; 124(5):1493-1505. doi: 10.1213/ANE.0000000000001707.
[2] Neff T et al. Start-Up Delays of Infusion Syringe Pumps; Paediatr Anaesth. 2001; 11(5):561-5. doi: 10.1046/j.1460-9592.2001.00730.x.
[3] Baeckert M et al. Performance of Modern Syringe Infusion Pump Assemblies at Low Infusion Rates in the Perioperative Setting; Br J Anaesth. 2020 February; 124(2):173-182. doi: 10.1016/j.bja.2019.10.007.
[4] Russotto V et al. Effect of central venous pressure on back-flow and bolus events during vasopressor syringe changeover. Br J Anaesth. 2020 December; 125(6):e463-e464. doi: 10.1016/j.bja.2020.09.005. Epub 2020 Sep. 25.
[5]) Elli S et al. Changing the syringe pump: A challenging procedure in critically ill patients. J Vasc Access. 2020 November; 21(6):868-874. doi: 10.1177/1129729820909024. Epub 2020 Mar. 4.
[6] Moss D R et al. An In Vitro Analysis of Central Venous Drug Delivery by Continuous Infusion: The Effect of Manifold Design and Port Selection. Anesth Analg. 2009 November; 109(5):1524-9. doi: 10.1213/ANE.0b013e3181 b7c359.
[7] Cour M et al. Benefits of smart pumps for automated changeovers of vasoactive drug infusion pumps: a quasi-experimental study. Br J Anaesth. 2013 November; 111(5):818-24. doi: 10.1093/bja/aet199. Epub 2013 Jun. 11.
[8]) Tsao A C et al. Delivery interaction between co-infused medications: an in vitro modeling study of microinfusion. Paediatr Anaesth. 2013 January; 23(1):33-9. doi: 10.1111/j.1460-9592.2012.03898.x. Epub 2012 Jun. 20.
[9] Parker M J et al. Novel Pump Control Technology Accelerates Drug Delivery Onset in a Model of Pediatric Drug Infusion. Anesth Analg. 2017 April; 124(4):1129-1134. doi: 10.1213/ANE.0000000000001706.
[10] Hadaway L. Stopcocks for Infusion Therapy: Evidence and Experience. J Infus Nurs. January/February 2018; 41(1):24-34. doi: 10.1097/NAN.0000000000000258.
[11] Muffly M K et al. Small-Volume Injections: Evaluation of Volume Administration Deviation From Intended Injection Volumes. Anesth Analg. 2017 October; 125(4):1192-1199. doi: 10.1213/ANE.0000000000001976.
[12] Yamaguchi K et al. A simulation study of high-flow versus normal-flow three-way stopcock for rapid fluid administration in emergency situations: A randomised crossover design. Australian Critical Care. 2021 Apr. 26, ISSN 1036-7314, https://doi.org/10.1016/j.aucc.2021.01.008.
[13] Kim D W, Stewart D J. The effect of syringe size on the performance of an infusion pump. Paediatr Anaesth. 1999; 9(4):335-7. doi: 10.1046/j.1460-9592.1999.00402.x.
[14] Kawakami H et al. Amount of accidental flush by syringe pump due to inappropriate release of occluded intravenous line. Technol Health Care. 2013; 21(6):581-6. doi: 10.3233/THC-130754.

### SUMMARY OF THE INVENTION

One aspect of the invention relates to a fluid control device which overcomes the disadvantages of the prior art. Another aspect relates to a device which, for changing syringe pump infusions, allows the infusions to be safely and easily connected to a carrier infusion in the continuous flow of the desired flow rate and/or changed for a running, soon to be empty syringe pump infusion without having to overcome dead spaces. Accordingly, the invention also relates to a device in which the dead space to the infusion flow can be easily overcome or eliminated during an injection or aspiration through/from an attachment nozzle of an infusion line in order to avoid or reduce the above-mentioned disadvantages and risks.

in an embodiment, the medical fluid control device for changing and controlling fluid flows, in particular in medical infusions, comprises a base body and an actuator body which is movable relative to the base body. The base body and the actuator body have, respectively, a sliding and sealing surface facing each other and the actuator body is fastened movably to the base body.

The actuator body has at least one actuator fluid channel, at least one actuator attachment nozzle for attaching hoses or syringes, and at least one actuator opening in the sliding and sealing surface of the actuator body.

Each actuator fluid channel in each case connects an actuator attachment nozzle directly to an actuator opening in the sliding and sealing surface of the actuator body. In other words, there are no other actuator fluid channels in the actuator body that do not connect an actuator attachment nozzle to an actuator opening in the sliding and sealing surface of the actuator body. Accordingly, the valve body does not have any closed dead spaces or dead volumes in any position, which cannot be flushed or can only be flushed with difficulty when the valve body is adjusted.

The base body has a first base fluid channel and a second base fluid channel within each case a base attachment nozzle. The first base fluid channel and the second base fluid channel are independent of each other and are not directly fluidically connectable to each other via the actuator body. In other words, the base fluid channels are separate from each other in the fluid control device and cannot be directly connected to each other within the fluid control device. In addition, the first base fluid channel and the second base fluid channel each comprise a base opening in the sliding and sealing surface of the base body and are free of a branching in the base body. This means that the base fluid channel has no branches and therefore also has no areas such as dead spaces or dead volumes that cannot be flushed or can only be flushed with difficulty, depending on the position of the actuator.

The actuator body is movable in such a way that the at least one actuator fluid channel can selectable be brought into a closed position or can be brought into an open position in connection with the first or the second base fluid channel. In other words, the respective openings in the sliding and sealing surface of the actuator body and the base body can be brought into alignment in order to connect the actuator fluid channel to the first or the second base fluid channel. In the closed position, the actuator opening of the actuator fluid channel is closed by the sliding and sealing surface of the base body. A base opening of a base fluid channel can also be closed by the sliding and sealing surface of the actuator body, depending on the position of the actuator body.

The sliding and sealing surfaces of the base body and the actuator body provide sufficient tightness so that no separate sealing element is required. In other words, a fluid control device can be constructed without separate sealing elements between the base body and the actuator body, in particular without sealing rings in the area of the actuator and/or base opening.

With these basic principles, fluid control devices with different geometric designs can be realized as single or multiple systems (so-called multi-fold systems) and for different applications. Some fluid control devices can have a single control port or they can have two control ports. However, the fluid control devices always have two base fluid channels, one of which is typically used as a drainage system for flushing the at least one actuator fluid channel. With regard to the other base fluid channel, which is typically used as an infusion channel, the medical fluid control devices described can be divided into two functional groups: (A) fluid control devices without interruption of an infusion channel when a new inflow port is switched on and (B) fluid control devices with interruption of the infusion channel when the new inflow port is switched on.

The fluid control device allows an infusion line to be connected via the first base attachment nozzle and at the same time has a fluidically separated drainage system via the second base attachment nozzle. A syringe pump newly connected to an actuator attachment nozzle of the actuator body can be guided via the drainage system during start-up. Not only an initial priming bolus but also additional liquid boluses can be delivered into the drainage system for the start-up. However, the delivery of a liquid bolus or even repeated liquid boluses leads to a strong compression or stretching of the components of the syringe pump system to be started up with the pressure values generated, which spring back after the end of the bolus and have to be compressed or stretched again until the steady-state flow rate is reached, which contributes to the start-up delay. To avoid this, a higher flow rate (e.g. 5 ml/h) than the final desired flow rate can be selected initially and the pump started. Higher flow rates from syringe pumps overcome start-up delays more quickly. The flow rate is then slowly and continuously reduced to the final desired flow rate (e.g. 1 ml/h) at the syringe pump.

Once the newly started syringe pump infusion has reached continuous flow conditions at the outflow system, the fluid control device allows the new syringe pump infusion to be connected from the outflow system to the infusion system with a movement of the actuator body. Depending on the design of the fluid control device, a syringe pump that is about to be emptied can be switched from the infusion line to the outflow system at the same time. In particular, with a double-port actuator, the fluid control device can be used to exchange an old (soon to be emptied) and a new (freshly started) syringe pump with a single movement of the actuator body. In an intermediate position, both openings in the sliding and sealing surface of the actuator body can be closed with respect to the base body. Devices with double-port actuator bodies make it possible to deliver the medication from the newly connected syringe pump at the exact point in the infusion system where the stopped syringe pump previously delivered it. This avoids gaps and duplications in the corresponding drug delivery (multi-infusion problem) when the syringe pump is changed.

The fluid control device can be used by not exchanging an old (soon to be emptied) and a new (freshly started) syringe pump, but by connecting the new, freshly started syringe pump to a single port, which initially drains into the outflow system and is then switched from the outflow system to the infusion line under steady-state flow conditions by changing the position of the actuator body. In an intermediate position, the channel can be closed. The fluid control device, especially if it is connected in series several times to an infusion line or is designed as a multi-fold system (see below), allows several individual syringe pumps to be connected freely and independently to a carrier infusion system and, if necessary, the soon empty syringe pump to be left in place until the patient or their circulation has proven to be stable before the empty syringe pump is finally disconnected.

The single and double port versions not only allow syringe pumps and other infusions to be connected without dead space or changed without major flow fluctuations, but also enable syringes to be attached for precise injection of medication and fluids as well as aspiration of blood or fluids from a catheter, infusion or tubing system. In addition, the described fluid control devices considerably reduce the resistance to the flow of fluids compared to known three-way valves, since the latter have fluid channels within the actuator body that are usually smaller in diameter than in the base body. The fluid control devices described have further decisive advantages over conventional three-way valves: Air or drug residues in the attachment nozzle (port) and/or in the infusion tube/syringe cone can first be discharged (drained/flushed out) into the drainage system (e.g. the second base fluid channel) before the actual infusion or precise injection into the infusion line (e.g. the first base fluid channel) then takes place. After the injection or aspiration, the connecting piece is turned or pushed back onto the drainage system and the remaining contents are expelled or rinsed out with NaCl 0.9%. This increases safety against air and other embolisms as well as unintentional injection of medication from the three-way stopcock cone and precipitation of medication due to physical incompatibility in the three-way stopcock cone. Flushing the cone and dead space into the drainage system instead of through the infusion line into the patient also reduces the patient's fluid load and protects the patient from unwanted late effects of medication from the dead space.

Furthermore, with the fluid control device without interrupting the infusion line it is not possible to block the carrier infusion—with other upstream infusions or syringe pumps if necessary—unintentionally (accidental occlusion) and thus also not to generate a corresponding excess pressure bolus or to release this unintentionally from the infusion line into the catheter.

Medical fluid control devices are often disposable products made of plastic and include valves, valve systems, three-way stopcocks or stopcocks.

Preferred embodiments of the invention are disclosed herein.

In some embodiments, the at least one actuator fluid channel, the first base fluid channel and the second base fluid channel can have the same cross-section. It is also possible to design the fluid channels with different cross-sections.

In some embodiments, the actuator body can be rotatable through 360° about an axis of rotation and has a rotationally symmetrical sliding and sealing surface. The actuator and base openings can be arranged in such a way that the connection between the actuator and base fluid channels can be changed with a 180° rotation.

In some embodiments, the first and second base openings can be arranged in the sliding and sealing surface at a distance of 180° from one another with the same radius relative to the axis of rotation, so that the at least one actuator opening of the actuator body can be moved from the first to the second base opening with a 180° rotation.

In some embodiments, the actuator body can be designed in the form of a rotatable disk with a circular sliding and sealing surface. The base body can have a correspondingly complementary circular sliding and sealing surface. The base body can also be designed as a disk or as a block with circular recesses for the actuator(s). In the case of a disk, the actuator and base attachment nozzle can be arranged vertically on the disk, namely on the side opposite the sliding and sealing surface. In the case of a block-shaped design of the base body, the base attachment nozzle can also be arranged laterally, i.e. perpendicular to the axis of rotation of the actuator body, or also vertically on the side opposite the sliding and sealing surface parallel to the axis of rotation.

In some embodiments, the actuator body can be designed in the form of a circular rotatable sleeve with the sliding and sealing surface on the inside. The base body can have a corresponding complementary sliding and sealing surface on a circumferential surface of a circular cylinder. The at least one actuator attachment nozzle can then be arranged perpendicular to the axis of rotation on the outer surface of the sleeve. The base attachment nozzles can be arranged parallel to the axis of rotation on the end faces of the circular cylinder.

In some embodiments, the actuator body can be designed in the form of a rotatable circular cylinder with the sliding and sealing surface on the lateral surface. The base body can have a corresponding recess with a sliding and sealing surface complementary to the sliding and sealing surface of the actuator body. The at least one actuator attachment nozzle can then be arranged on the end face of the circular cylinder perpendicular to the axis of rotation.

In some embodiments, the actuator body can be designed in the form of a rotatable circular cylinder with the sliding and sealing surface on the end face, whereby areas of the lateral surface can also serve as the sliding and sealing surface. The base body can have a corresponding recess with a sliding and sealing surface complementary to the sliding and sealing surface of the actuator body. The at least one actuator attachment nozzle can then be arranged on the end face of the circular cylinder perpendicular to the axis of rotation. Alternatively, the actuator body can also be conical so that it tapers towards the sliding and sealing surface. This design, particularly in the conical version, allows medication to be applied to the same point or a limited, short section or chamber of the infusion channel (first base fluid channel), thereby reducing or avoiding the flow and dead space problems of the multi-infusion problem.

The versions with rotatable actuator bodies are particularly suitable for fluid control devices with two actuator attachment nozzles, whereby the connection between the actuator attachment nozzles and the base actuator attachment nozzles can be changed quickly and easily (so-called Quick-Exchange).

A fluid control device for changing and controlling fluid flows, in particular in medical infusions, comprising a base body and a plurality of actuator bodies movable relative to the base body; wherein the base body and the plurality of actuator bodies each have a sliding and sealing surface facing one another and the plurality of actuator bodies are movably attached to the base body, can be regarded as an independent invention. The respective actuator body can have at least one actuator fluid channel, at least one actuator attachment nozzle for connecting hoses or syringes and at least one actuator opening in the sliding and sealing surface of the actuator body. Each actuator fluid channel connects an actuator attachment nozzle directly to an actuator opening in the sliding and sealing surface of the actuator body. The base body can have a first base fluid channel (typically for an infusion line) and a second base fluid channel (typically for drainage), each with a base attachment nozzle, whereby the first base fluid channel and the second base fluid channel are independent of each other and cannot be fluidically connected directly to each other via the actuator body. The first base fluid channel can form a central chamber with several base openings. This is usually designed in such a way that it can be flowed through easily and completely without forming dead spaces. The second base fluid channel can also form a central chamber with several base openings, whereby the dead space problem plays a subordinate role here and a base channel with branches to the respective base openings would also be possible. The actuator body can be movable in such a way that the at least one actuator fluid channel can be brought either into a closed position or into an open position in connection with the first or second base fluid channel.

In some embodiments, the actuator body can be designed in the form of a linearly displaceable plate with the sliding and sealing surface. The base body can have a corresponding recess with a sliding and sealing surface complementary to the sliding and sealing surface of the actuator body.

The variant with linear sliding actuator body is suitable for fluid control devices with an actuator attachment nozzle that can be moved back and forth between the base fluid channels.

In some embodiments, the fluid control device can be a multi-fold system comprising a plurality of actuator bodies which can be moved relative to the base body and which are movably attached to the base body in a plurality of recesses. The actuator bodies can have the shapes mentioned above, whereby actuator bodies with different shapes can also be combined.

In some embodiments, the first base fluid channel and/or the second base fluid channel can have a second, additional base attachment nozzle, so that at least one base fluid channel forms a continuous base fluid channel in the base body with base attachment nozzles on both sides, irrespective of the position of the actuator body.

Such a continuous base fluid channel is suitable for connecting an infusion line with carrier solution, into which a drug solution can be connected via the actuator body as required.

In some embodiments, the continuous base fluid channel can be guided through the sliding and sealing surface of the base body, forming the base opening of the base fluid channel. This enables fluid channel routing with an opening in the sliding and sealing surface without forming a branch, which would form a dead space or dead leg that is difficult to flush, depending on the position of the actuator body.

In some embodiments, the base fluid channel, in particular the continuous base fluid channel, can be designed in such a way that it does not have a dead leg regardless of the position of the actuator body. For this purpose, the base fluid channel can be guided in the base body in such a way that it is open in the area of the sliding and sealing surface of the base body and thus forms the base opening. The base opening can extend along the sliding and sealing surface of the base body. In an open position, the actuator opening can be brought into alignment with the base opening, while part of the base opening is covered by the sliding and sealing surface of the actuator body. In a closed position, the base opening is completely covered by the sliding and sealing surface of the actuator body. By guiding the base fluid channels in this way, dead legs within the fluid control device can be avoided.

In other words, the continuous base fluid channel within the base body does not have a branch and can always be completely flushed. A branch only occurs within the fluid control device when the actuator fluid channel is connected to the corresponding base opening in the base body.

In some embodiments, the actuator body may have a single actuator fluid channel and the base body may have more than two base fluid channels. The additional base fluid channels can be used as infusion channels for aspirating fluids or medications into the syringe or ejecting them from the syringe.

This arrangement makes it possible, for example, to aspirate blood from a catheter, eject blood or other fluids into the drainage system, eject blood into a blood collection tube or an analyzer, aspirate NaCl 0.9% from an infusion line and flush a selected port or aspirate contrast medium from a supply line and inject it through the catheter connection port to the catheter without having to remove/reattach an injection syringe from the injection device. In order to protect the syringe or injection port from unintentional injections or passive backflow, the injection port is placed in a closed position. Such a device is advantageous for invasive catheterization in angio or cardiac catheter laboratories, for manual or automated blood sampling for analyses, blood exchange, or for manual massive infusion of blood and fluids, for example. The advantage here is the low flow resistance of the device according to the invention as well as the fact that the syringe remains on the injection device (reduced risk of infection by avoiding repetitive reattachment of syringes) and a reduced fluid load on the patient, in which the cone of the injection port is not flushed into the patient but into the waste/drainage channel using NaCl 0.9%. In order to be able to completely empty the syringe itself (including the syringe cone lumen), the syringe must be constructed in such a way that the plunger has a spike in the syringe cone in order to empty it of air and liquid.

In some embodiments, the base body and the actuator body can each have complementary latching means, so that the actuator body can be latched in different positions.

In some embodiments, at least one base attachment nozzle and the at least one actuator attachment nozzle can be designed as a Luer connection.

In some embodiments, the sliding and sealing surfaces of the actuator body and the base body can be in sealing contact with each other.

In some embodiments, the fluid control device may be manufactured as a disposable article made of plastic.

In some embodiments, the first base fluid channel and/or the second base fluid channel can be designed with a pressure measuring device and/or a pressure regulating device. This can be realized, for example, by (i) the base body being designed with a connection for a pressure measuring system on the first base fluid channel and/or on the second base fluid channel, to which a pressure gauge can be connected or is connected, or (ii) the base body being designed with a pressure sensor on the first base fluid channel and/or on the second base fluid channel. In this way, the pressure in the individual channels or pressure differences between the infusion lumen and the newly attached infusion leg can be detected before switching on and brought to the desired level.

In some embodiments, the second base fluid channel can be designed with an adjustable or fixed pressure outlet valve. In this way, a newly attached syringe pump, which is initialized via the drainage, can be brought to the desired pressure before it is connected to the infusion line.

In some embodiments, the base body may be configured with a pressure equalization indicator between the first base fluid channel and the second base fluid channel.

In some embodiments, the base body can be designed with a pressure regulation from the first base fluid channel to the second base fluid channel.

In some embodiments, the base body may have an internal pressure transfer from the infusion channel to the drainage system.

Such an embodiment of a fluid control device with a pressure measuring device and/or a pressure regulating device can be regarded as a separate invention and independent of the fluid control device described above.

The pressure in the drainage/outflow system (second base fluid channel) should preferably correspond to the pressure in the infusion channel/infusion line (first base fluid channel), so that when the newly started syringe pump, which is already delivering under steady-state flow conditions, is switched on, acute forward or backward fluid shifts between the infusion line and the newly connected syringe pump do not occur, which consequently leads to undesirable flow changes.

The desired pressure can be set as follows:

A) By positioning the medical fluid control device vertically above the patient's heart level, the pressure in the infusion channel can be reduced to zero and thus brought to the same level as the atmospheric pressure at the outlet of the drainage channel. For a corresponding vertical adjustment of the device, it is designed for a dockable pressure measurement display device or with an integrated pressure measurement display device.

B) As an alternative to absolute pressure measurement for vertical positioning of the medical fluid control device according to the invention above patient heart level for pressure equalization between the infusion channel and the drainage channel, it is designed with a pressure equalization indicator between the infusion channel and the drainage channel, which allows the vertical position to be adjusted so that the pressure between these two compartments is equal.

C) As an alternative to vertical positioning of the medical fluid control device according to the invention above the patient's heart level for pressure equalization between the infusion channel and the outflow channel, the pressure in the outflow channel is adapted by means of an adjustable or fixed pressure regulating valve at the outlet of the outflow system or by corresponding vertical positioning of the reservoir system connected to the outflow system. The level of pressure in the infusion line can usually be estimated based on the level of the central venous pressure or it can be determined directly, as described above. An additional pressure measurement in the drainage channel makes it possible to monitor the pressure in the drainage channel in a controlled manner.

D) As an alternative to the above pressure equalization techniques, the medical fluid control device according to the invention has a coupled, direct pressure regulation, whereby the pressure in the infusion channel controls the pressure valve at the outlet of the drainage system.

The medical fluid control device therefore not only makes it possible to bring newly connected syringe pumps to the set flow rate before switching them on, but also to compensate for pressure differences between the infusion line and the new infusion to be connected due to central venous pressure, resistances and high flow rates in the infusion line system, as well as due to infusion syringe pumps placed at different vertical heights, or to adapt them to the pressure of the infusion channel.

The application of the device according to the invention and its embodiments is not only limited to medical infusion lines/systems in the classical sense of infusion and drug delivery systems (injection), but can also be used in medical apparatuses with or in their line, pump, fluid delivery systems (e.g. extracorporeal membrane oxygenation (ECMO), cardiopulmonary bypass). in their line, tube and hose systems such as hose, pump, fluid delivery systems (e.g. extracorporeal membrane oxygenation (ECMO), cardiopulmonary bypass (CPB)), filter systems (dialysis, filtration, oxygenators), analysis systems, etc. for venting, injection, aspiration and for connecting medical infusion pumps, etc.

In its simplest form, the fluid control device can be a stopper or infusion stopper with an attachment nozzle on the base body and an attachment nozzle on the actuator body. The base body and the actuator body are then preferably designed as a disk. The stopper or infusion stopper can be connected upstream and/or downstream of the above embodiments and can therefore not only be used as a flow stopper with minimal resistance, but can also be used to direct the infusion flow towards or away from the patient. This simplest form of the fluid control device as a stopper or as an infusion stopper is not covered in the above embodiments, but can be regarded as an independent invention.

### BRIEF DESCRIPTION OF THE DRAWINGS

The invention will be explained in more detail below by means of embodiment examples in connection with the drawing(s). It shows:

FIGS. 1A-1D a fluid control device with two disks for quickly changing syringe pumps, under (a) a perspective view of the fluid control device (FIG. 1A), under (b) a sectional view (FIG. 11B), under (c) a perspective view of the base body (FIG. 1C), and under (d) a perspective view of the actuator body (FIG. 1D);

FIGS. 2A-2C a fluid control device with a block-like base body, under (a) a perspective view of the fluid control device (FIG. 2A), under (b) a perspective view of the actuator body (FIG. 2B), and under (c) a perspective view of the base body (FIG. 2C);

FIGS. 3A-3B a fluid control device with a continuous base fluid channel and an actuator body with two actuator fluid channels, under (a) a perspective view of the fluid control device (FIG. 3A), under (b) a perspective view of the base body (FIG. 3B);

FIGS. 4A-4B a fluid control device with a continuous base fluid channel and an actuator body with only one actuator fluid channel, under (a) a perspective view of the fluid control device (FIG. 4A), under (b) a perspective view of the base body (FIG. 4B);

FIGS. 5A-5C a fluid control device with a linearly displaceable actuator body, under (a) a perspective view of the fluid control device (FIG. 5A), under (b) a perspective view of the actuator body (FIG. 5B), and under (c) a perspective view of the base body (FIG. 5C);

FIG. 6 a fluid control device as a multi-fold system with several actuator bodies, under (a) a perspective view of the fluid control device;

FIGS. 7A-7B a fluid control device as a multi-fold system with a drainage chamber, under (a) a perspective view of the fluid control device (FIG. 7A), under (b) a sectional view (FIG. 7B);

FIGS. 8A-8D a fluid control device as a multi-fold system with circular cylinder-shaped actuator bodies, under (a) a perspective view of the fluid control device (FIG. 8A), under (b) a sectional view (FIG. 8B), under (c) a perspective view of the base body (FIG. 8C), and under (d) a perspective view of the actuator body (FIG. 8D);

FIGS. 9A-9D a fluid control device as a multi-fold system with a circular cylinder-shaped base body and sleeve-shaped actuator bodies, under (a) a perspective view of the fluid control device (FIG. 9A), under (b) a sectional view (FIG. 9B), under (c) a top view of the base body (FIG. 9C), and under (d) a perspective view of the actuator body (FIG. 9D);

FIG. 10A-10F a fluid control device as a multi-fold system with a cylindrical base body and laterally recessed cylindrical actuator bodies, under (a) a perspective view (FIG. 10A), under (b) a sectional view through the first base fluid channel (FIG. 10B), under (c) a sectional view through both base fluid channels (FIG. 10C), under (d) a sectional view through the second base fluid channel (FIG. 10D), under (e) a side view of the base body; under (f) a perspective view of the actuator body (FIG. 10E);

FIGS. 11A-11B a further variant of a fluid control device as a multi-fold system with a cylindrical base body and laterally recessed cylindrical actuator bodies, under (a) a perspective view (FIG. 11A), under (b) a sectional view through both base fluid channels (FIG. 11B);

FIGS. 12A-12D a fluid control device with more than two base fluid channels and a circular cylinder-shaped actuator body, under (a) a perspective view of the fluid control device (FIG. 12A), under (b) a sectional view (FIG. 12B), under (c) a perspective view of the base body (FIG. 12C), and under (d) a perspective view of the actuator body (FIG. 12D);

FIG. 13 a perspective view of a fluid control device with more than two base fluid channels and a disk-shaped actuator body;

FIG. 14 an exploded perspective view of a fluid control device with more than two base fluid channels and a disk-shaped actuator and base body;

FIGS. 15A-15E a perspective view of an infusion stopper with two disks rotatable relative to each other, under (a) a perspective view of the infusion stopper in an open position (FIG. 15A), under (b) a sectional view (FIG. 15B), under (c) a perspective view of the infusion stopper in a closed position (FIG. 15C), under (d) a perspective view of the base body (FIG. 15D), and under (e) a perspective view of the actuator body (FIG. 15E);

FIG. 16 a perspective view of a fluid control device with a circular cylinder-shaped actuator body and a sleeve-shaped base body;

FIGS. 17A-17B a fluid control device as a multi-fold system with a base body consisting of several sleeve-shaped segments and laterally embedded cylindrical actuator bodies, under (a) a perspective view (FIG. 17A), under (b) a side view of the base body (FIG. 17B);

FIG. 18 a fluid control device with pressure measuring device.

### EMBODIMENTS OF THE INVENTION

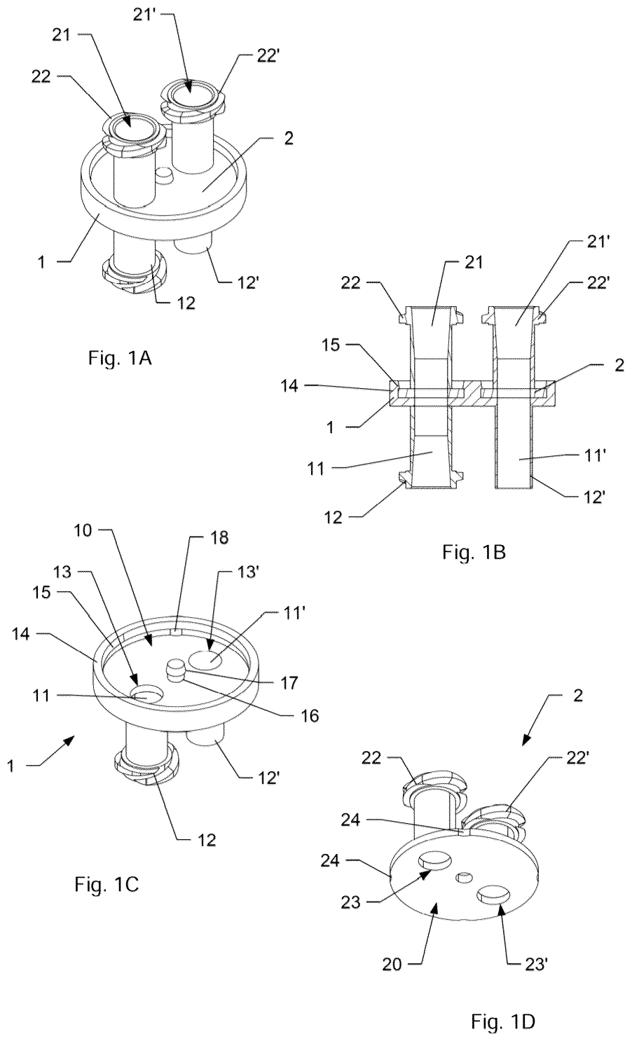

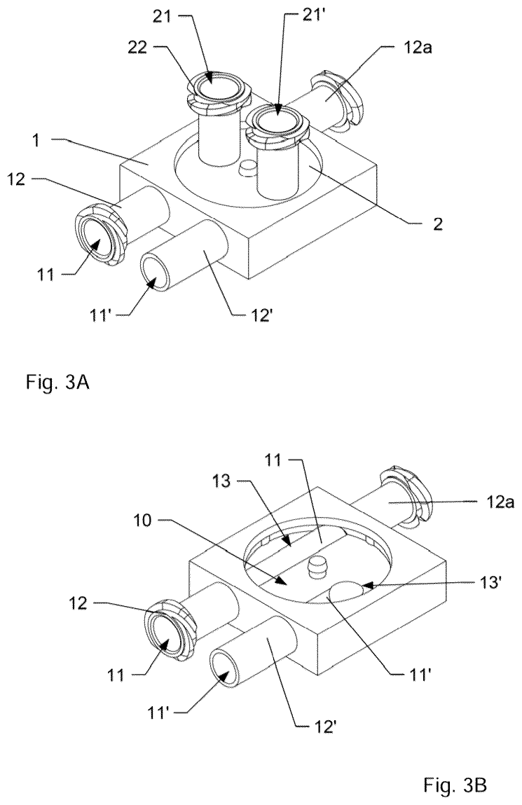

The medical fluid control devices described below can be divided into two functional groups: (A) fluid control devices without interruption of an infusion channel when a new inflow port is switched on (FIGS. 3A-3B, 4A-4B, 5A-5C, 6, 7A-7B, 8A-8D, 9a-9D, 10A-10F, 11A-11B, 16, 17A-17B) and (B) fluid control devices with interruption of the infusion channel when the new inflow port is switched on (FIGS. 1A-1D, 2A-2C, 12A-12D, 13, 14).

FIGS. 1A-1D show an embodiment of the medical fluid control device for changing and controlling fluid flows. FIG. 1A shows a perspective view and FIG. 1B shows a sectional view of the fluid control device. The fluid control device comprises a base body 1 (FIG. 1C) and an actuator body 2 (FIG. 1D), wherein the base body 1 and the actuator body 2 each have a sliding and sealing surface 10, 20 facing each other and the actuator body 2 is held in the base body 1 in a sealing and movable manner. A reverse arrangement is also possible.

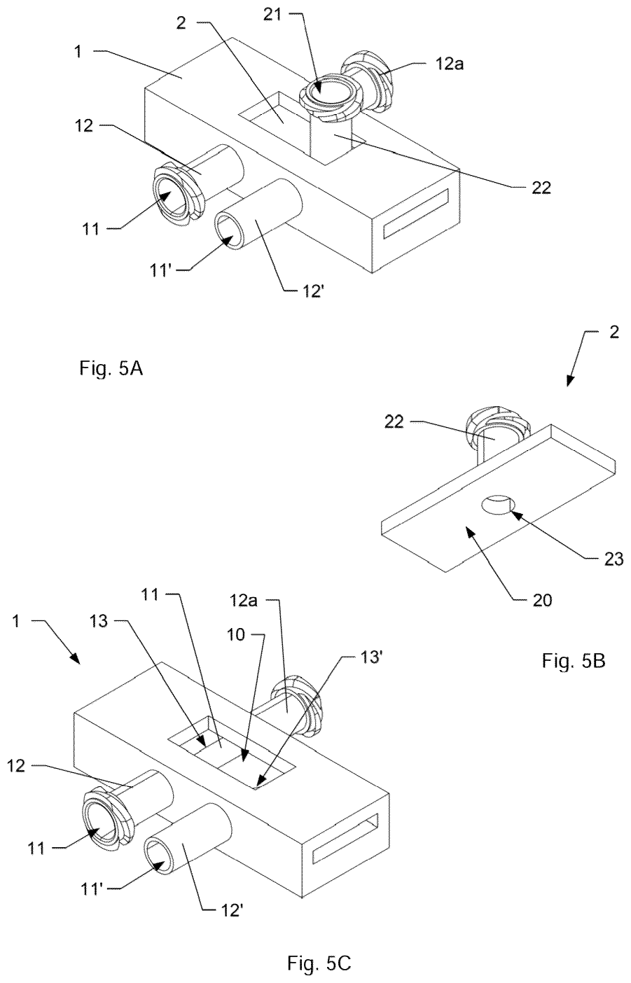

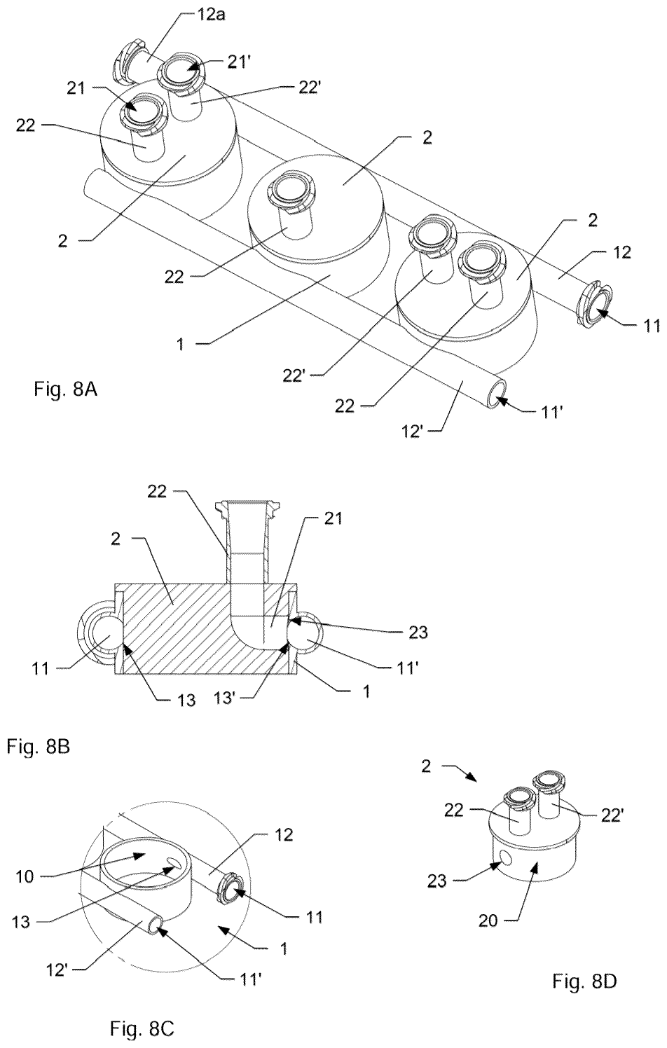

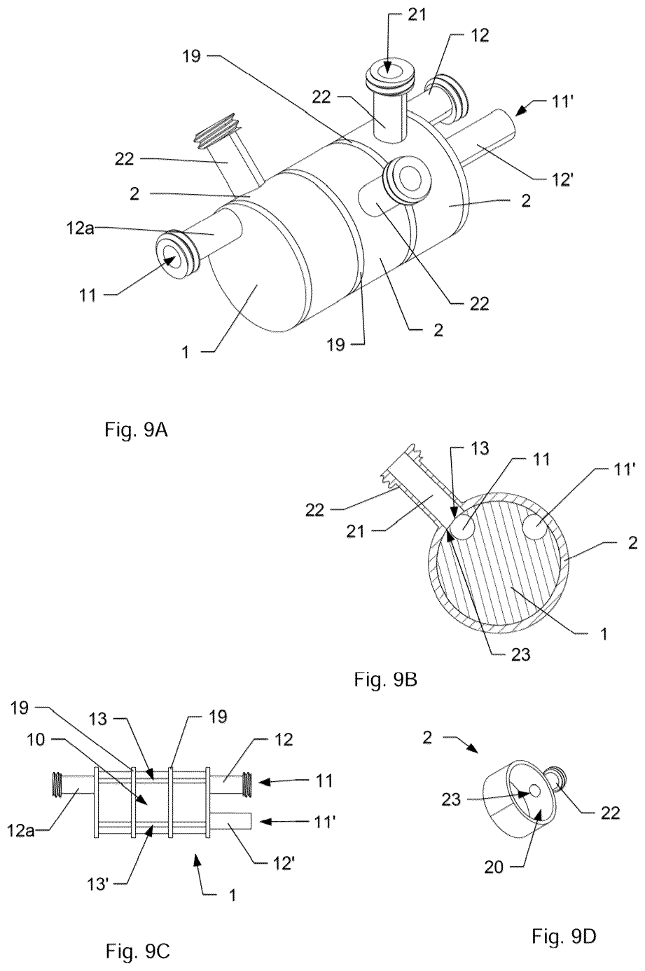

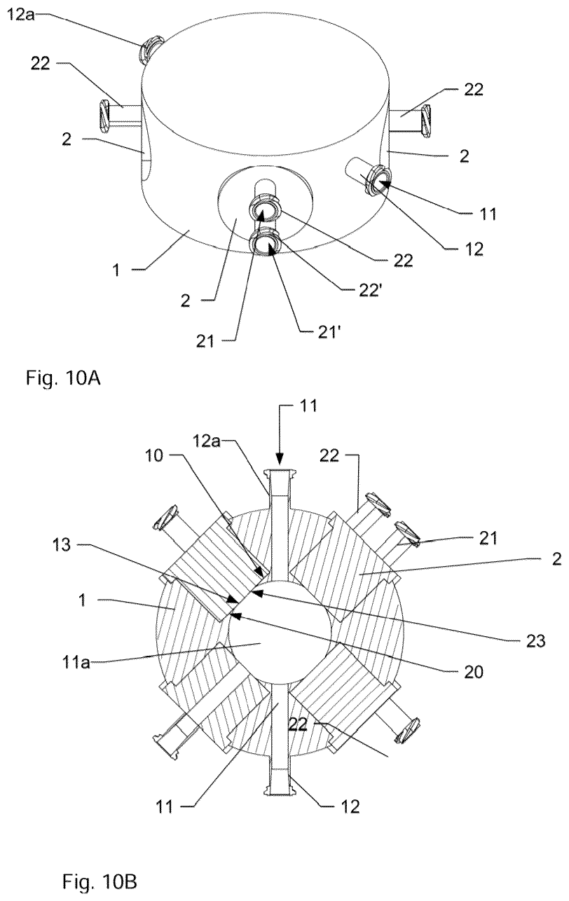

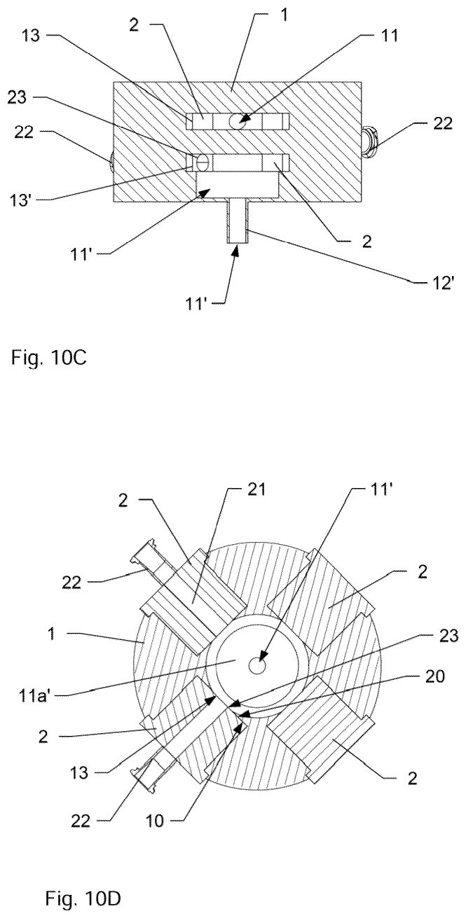

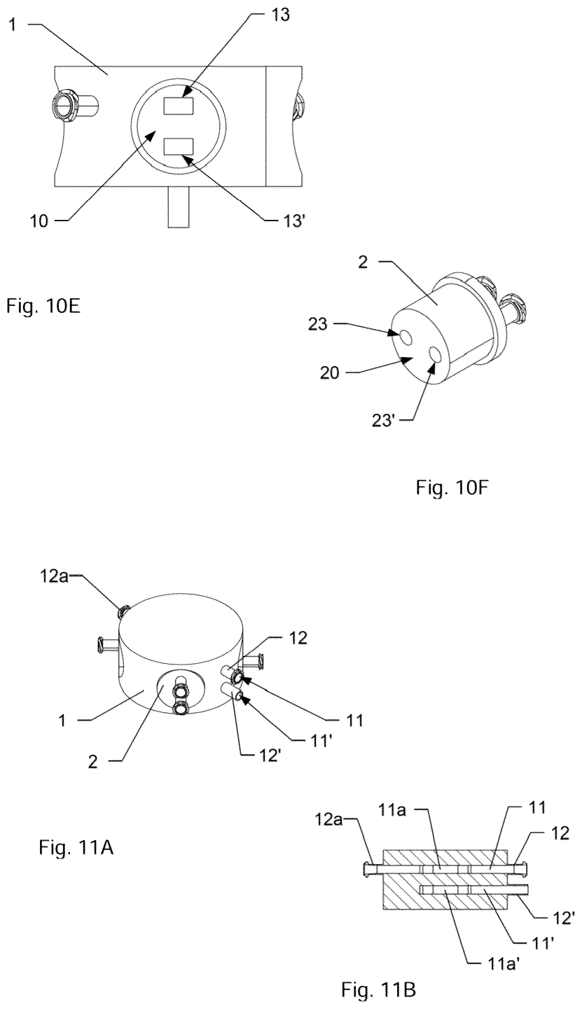

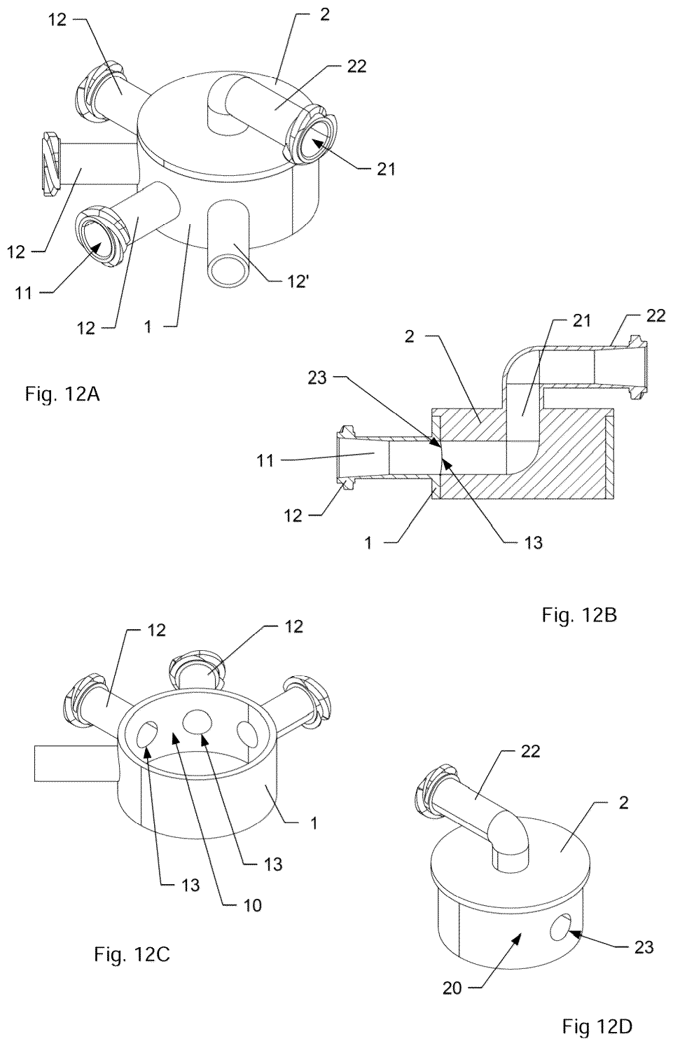

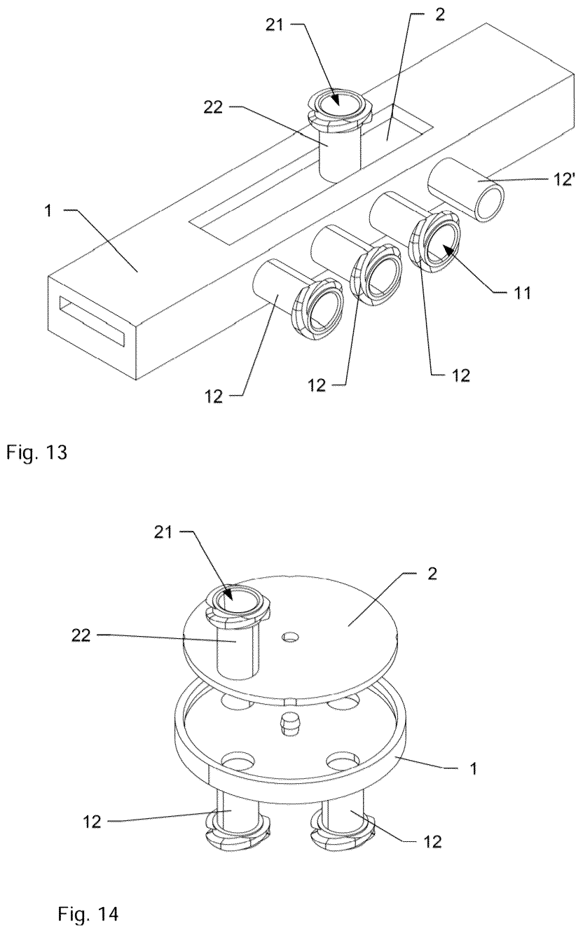

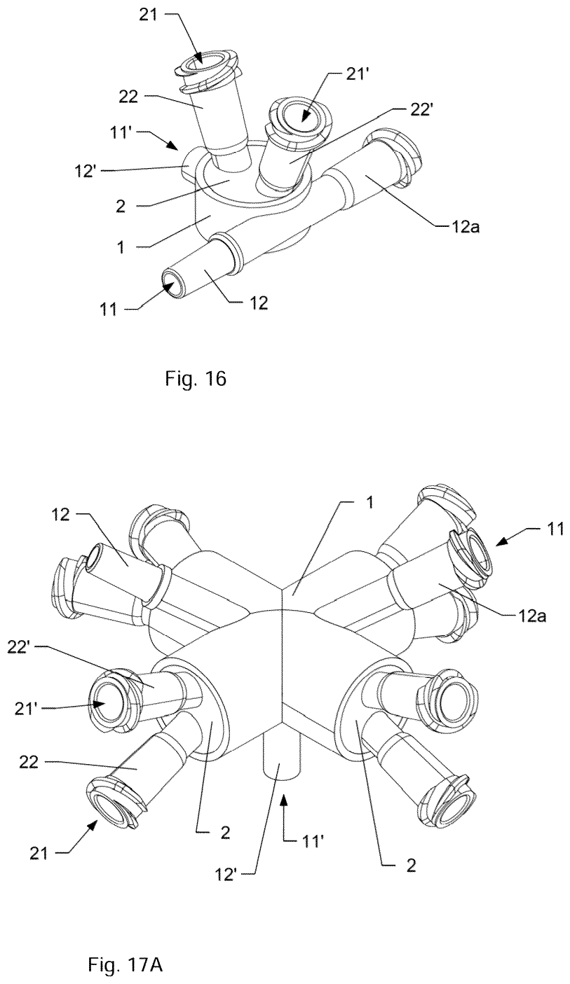

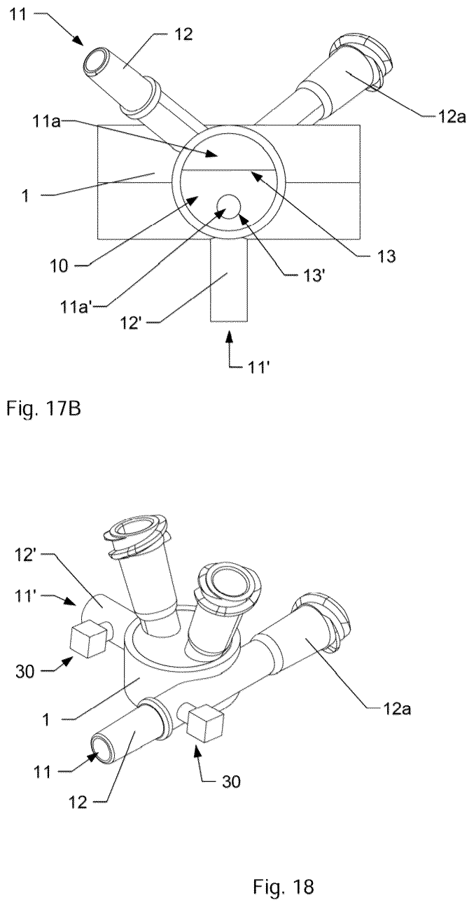

In the embodiment shown, the actuator body 2 is designed as a rotatable disk with a circular sliding and sealing surface 20. Other variants, e.g. a linearly displaceable plate (FIGS. 5A-5C, FIG. 13), a rotatable cylinder (sliding and sealing surface on the lateral surface) (FIG. 8, FIG. 12, FIG. 16), a rotatable cylinder (sliding and sealing surface on the lateral and end surfaces) (FIG. 10, FIG. 11, FIG. 17) or a rotatable sleeve (sliding and sealing surface on the inner surface) (FIG. 9) are also possible and are described in connection with other embodiments in the other figures. The sliding and sealing surface 10 of the base body 1 is correspondingly complementary to the sliding and sealing surface 20 of the actuator body 2. In the embodiment shown, the base body 1 is also designed as a disk with a circumferential edge 14.

In the embodiment of the fluid control device shown in FIGS. 1A-1D, the actuator body 1 has two separate actuator fluid channels 21, 21′, each with an actuator attachment nozzle 22, 22′. Both actuator fluid channels 21, 21′ each end in an actuator opening 23, 23′ in the sliding and sealing surface 20 of the actuator body 2. The actuator fluid channels 21, 21′ are independent of each other and cannot be fluidically connected to each other via the base body 1.

In the embodiment shown, the base body 1 has two separate base fluid channels 11, 11′, each with a base attachment nozzle 12, 12′. One base opening 13, 13′ of each of the two base fluid channels 11, 11′ is located in the sliding and sealing surface 10 of the base body 1. The base fluid channels 11, 11′ are independent of each other and cannot be fluidically connected to each other via the actuator body 2.

The actuator body 2 can be rotated about an axis of rotation A running through the center of the circular disc. The actuator attachment nozzles 22, 22′ of the actuator body 2 are parallel to the axis of rotation A, and their actuator openings 23, 23′ are arranged symmetrically at a distance of 180° around the axis of rotation A. The base attachment nozzles 12, 12′ of the base body 1 are also parallel to the axis of rotation A and are arranged with their base openings 13, 13′ symmetrically around the axis of rotation A, so that they can be brought into alignment with the actuator openings 23, 23′.

In a first open position (FIG. 1A and FIG. 1B), the first actuator fluid channel 21 is connected to the first base fluid channel 11 and the second actuator fluid channel 21′ is connected to the second base fluid channel 11′. By rotating the actuator body 2 by 180°, the connections of the fluid channels can be changed very quickly, so that the first actuator fluid channel 21 is connected to the second base fluid channel 11′ and the second actuator fluid channel 21′ is connected to the first base fluid channel 11. A rotation of 90° leads to a closed position in which the respective openings 13, 13′, 23, 23′ lie directly on the sliding and sealing surface of the counterpart and are closed. There is no dead volume, as is the case with a conventional stopcock, for example.

On the side of the sliding and sealing surface 10, the base body 1 has a wall 14 surrounding it with a radially inwardly directed projection 15. At the axis of rotation, the base body 1 also has a protruding pin 16 provided with latching means 17. The disk of the actuator body 2 is designed in such a way that it can engage under the projection 15 of the wall 14 and on the latching means 17 of the pin 16 and in this way is held in the base body 1 in a sealing and rotatable manner. Furthermore, the wall 14 of the base body 1 has one or more latching lugs 18 on the inside, for example arranged in 90° steps around the axis of rotation. The actuator body 2 has corresponding latching notches 24 with which the actuator body 2 can latch into the various positions.

Such a fluid control device is particularly suitable for connecting e.g. syringe pumps to an infusion line and for quickly changing syringe pumps (so-called quick exchange).

For example, an outgoing infusion line (infusion channel) to a patient can be connected to the first attachment nozzle 12 (connection port) of the base body 1, while the second attachment nozzle 12′ is connected to an outgoing drainage line (drainage channel), for example to a collection bag. A supply infusion line, e.g. from an infusion pump, can now be connected to each of the two attachment nozzles 22, 22′ of the actuator body 2 (port A and port B).

For example, an old, soon to be empty syringe pump infusion can infuse into the infusion channel (base fluid channel 11; actuator attachment nozzle 22) via port A (actuator fluid channel 21; base attachment nozzle 12), while the new, full syringe pump infusion infuses into the drainage channel (base fluid channel 11′; base attachment nozzle 12) via port B (actuator fluid channel 21′; actuator attachment nozzle 22′). The drainage channel can either be connected to an aspiration unit or a capacitive collection unit. The drainage access allows the syringe pump line and connection points to be kept free of air and drug residues and, with the manual or automated administration of a fluid bolus from the syringe, the fluid delivery from the syringe pump can be brought up to the desired continuous delivery rate more quickly.

As soon as the new, full syringe pump infusion is at the desired continuous delivery rate, the two ports with the infusion accesses are changed by rotating the actuator body through 180°. The new, full syringe pump infusion is now connected to the infusion channel and the old, soon to be empty syringe pump infusion is connected to the drainage channel, from which it can now be disconnected and prepared for another change.

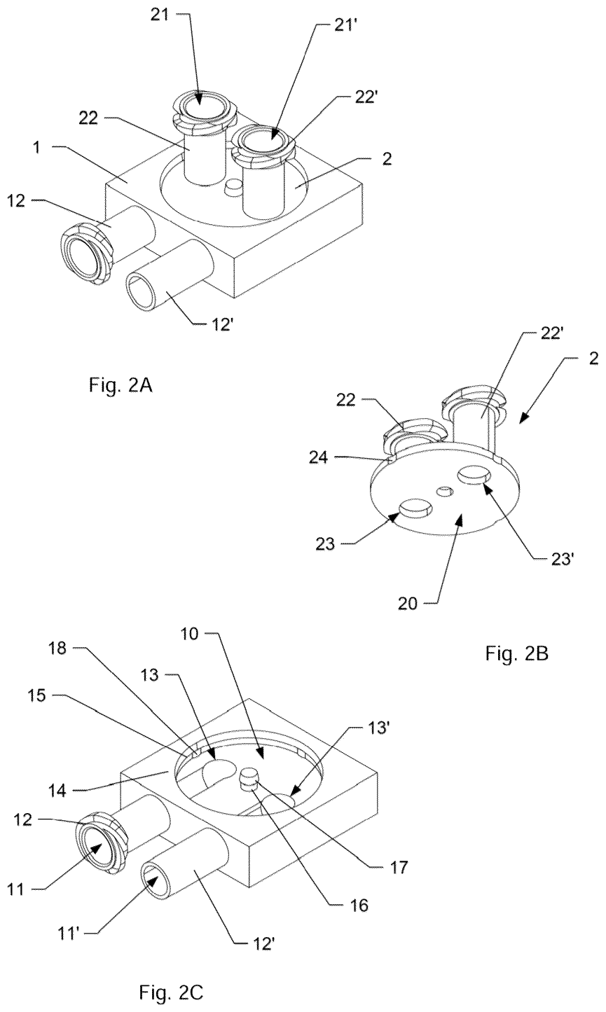

FIGS. 2A-2C show a further embodiment of a fluid control device, which is also suitable for a quick exchange. In contrast to the fluid control device shown in FIGS. 1A-1D, the base body 1 is not designed as a disk, but as a block. The two base attachment nozzles 12, 12′ are arranged side by side. Accordingly, the two base fluid channels 11, 11′ have a right angle. The disk-shaped actuator body 2 is accommodated in a circular recess in the base body 1. The mode of operation of the fluid control device shown in FIG. 2 is the same as that of the fluid control device shown in FIGS. 1A-1D.

The base opening 13, 13′ of the base fluid channel 11, 11′ can—as shown in FIG. 2(c)—extend along the base fluid channel 11, 11′ in the sliding and sealing surface 10 and lie open. In this case, the sliding and sealing surface 20 of the actuator body 2 partially forms a wall of the base fluid channel 11, 11′. The function of such an exposed base fluid channel 11, 11′ is described in detail in the following embodiments. Alternatively, the base fluid channel 11, 11′ can be covered by the sliding and sealing surface 10, so that only the round base opening 13, 13′, which is complementary to the actuator opening 23, 23′, is located in the sliding and sealing surface of the base body 1.

FIGS. 3A-3B show a further embodiment of a fluid control device, which is also suitable for a quick exchange and also has a block-like base body as in FIGS. 2A-2C. In contrast to the fluid control devices in FIGS. 1A-1D and FIGS. 2A-2C, the fluid control device in FIGS. 3A-3B has a continuous base fluid channel 11, which ends in an additional base attachment nozzle 12a. One of the two base fluid channels 11 correspondingly has two base attachment nozzles 12, 12a. However, the two base fluid channels 11, 11′ are also separate from each other and cannot be fluidically connected to each other via the actuator body 2.

In the case of a base fluid channel 11 with two base attachment nozzles 12, 12a, this continuous base fluid channel 11 breaks through the sliding and sealing surface 10 so that it is open and forms the base opening 13. In the embodiment shown, the base opening extends essentially along the entire sliding and sealing surface 10 of the base body 1 (see FIG. 3B). This ensures that a connected actuator fluid channel 21 opens directly into the continuous base fluid channel 11 and that there is no—even if only a small—side arm of the base fluid channel 11, which forms a dead leg depending on the position of the actuator body. There is therefore no space in the base body 1 in which air or deposits can accumulate. It is also possible to design both base fluid channels (11, 11′) as continuous fluid channels.

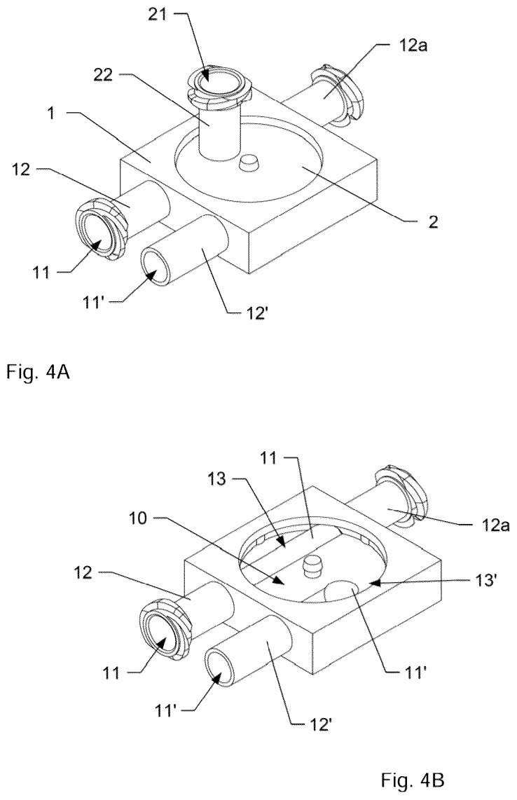

FIGS. 4A-4B show a fluid control device which, in contrast to the fluid control device in FIGS. 3A-3B, has an actuator body 2 with only one actuator fluid channel 21 and corresponding actuator attachment nozzle 22 and actuator opening 23. Such a fluid control device is not used for switching between two supply lines, but for dead space-free connection of a supply line or injection from a syringe into a base fluid channel.

In one application, for example, a new, full syringe pump infusion can be switched into a running infusion through the continuous base fluid channel 11. In this case, the new, full syringe pump is connected to the actuator attachment nozzle 22 of the actuator body. This first infuses via the single port into the non-continuous base fluid channel 11′ (drainage channel). The drainage channel can either be connected to a suction unit or a capacitive collection unit. It can also be continuous with two base connection nozzles and be flushed with a flushing solution, for example, i.e. it has an inlet and an outlet connection nozzle (see FIG. 8A). The drainage access allows the syringe pump line and connection points to be kept free of air and drug residues and, with the manual or automated administration of a bolus, the fluid delivery by the syringe pump can be brought to the desired continuous delivery rate more quickly. As soon as the new, full syringe pump infusion is at the desired continuous delivery rate, the actuator fluid channel 21 is moved from the drainage line (base fluid channel 11′) to the continuous infusion line (base fluid channel 11) by rotating the actuator body 1 by 180°. The new, full syringe pump infusion is now connected to the infusion channel.

Alternatively, a medication syringe can be connected instead of the syringe pump. The drainage access allows the syringe and connection points to be cleared of air and old fluid and medication residues and the medication in the syringe to be brought to the infusion level without dead space. By rotating the actuator body by 180°, the medication syringe is connected to the continuous base fluid channel 11. The syringe is now connected to the infusion line and the medication can be injected. The actuator 1 can then be rotated through 180° again, the medication syringe removed and the fluid channel 21 cleaned by injecting a syringe containing a rinsing solution (e.g. 0.9% NaCl solution) into the drainage channel (any remaining medication is expelled). A further rotation of the actuator body 1 by 90° brings the actuator fluid channel 21 into a closed position.

FIGS. 5A-5C show a fluid control device which, unlike the previous fluid control devices, is actuated by a linear displacement of the actuator body 2. In the embodiment shown, the actuator body 2 is designed as a displaceable, rectangular plate. The fluid control device shown in FIG. 5 has a single actuator fluid channel 21 with actuator attachment nozzle 22 and actuator opening in the sealing and sliding surface 20. In the variant shown, the actuator attachment nozzle is positioned vertically on the side of the plate opposite the sliding and sealing surface. The movable plate is accommodated in a corresponding recess in the base body 1. The base body 2 has the two base fluid channels 11, 11′ with corresponding base attachment nozzles 12, 12′, one of which is designed as a continuous base fluid channel 11 with a second base attachment nozzle 12a. As in the preceding embodiments, the continuous base fluid channel 11 is guided to break through the sliding and sealing surface. The base opening 13 of the continuous base fluid channel 11 thus extends in the sliding and sealing surface 10 along the base fluid channel 11 and is partially or completely open. Here too, the base fluid channels 11, 11′ do not have any branches or side arms within the base body 1. When the base opening is closed, the continuous base fluid channel 11 is completely flushed. As in the preceding embodiments, the non-through base fluid channel 11′ shown in FIG. 5 ends in the base opening 13′, which here has a complementary cross-section to the actuator opening 23 of the actuator body 2.

By moving the plate or the actuator body 2, the actuator fluid channel 21 can be connected to one of the two base fluid channels 11, 11′. A closed intermediate position, in which the actuator opening 23 is closed by the sliding and sealing surface 10 of the base body 1, is also possible.

This fluid control device is also suitable for connecting a syringe pump to an infusion line or for injecting medication from a syringe into an infusion line.

In one application, for example, a new, full syringe pump infusion can be switched into a running infusion through the continuous base fluid channel 11. In this case, the new, full syringe pump is connected to the actuator attachment nozzle 22 of the actuator body. This first infuses via the single port into the non-continuous base fluid channel 11′ (drainage channel). However, this can also be designed as a continuous and correspondingly flushable base fluid channel with two base attachment nozzles. The drainage channel can be connected either to a suction unit or a capacitive collection unit. The drainage access allows the syringe pump line and connection points to be kept free of air and drug residues and, with the manual or automated administration of one or more boluses, to bring the fluid delivery from the syringe pump more quickly to the desired continuous delivery rate. As soon as the new, full syringe pump infusion reaches the desired continuous delivery rate, the actuator fluid channel 21 is moved from the drainage line (base fluid channel 11′) to the continuous infusion line (base fluid channel 11) by moving the actuator body 1. The new, full syringe pump infusion is now connected to the infusion channel.

Alternatively, a medication syringe can be connected instead of the syringe pump. The drainage access makes it possible to free the syringe and connection points from air and old fluid and medication residues and to bring the medication in the syringe to the infusion level without dead space. By moving the actuator body, the medication syringe is connected to the continuous base fluid channel 11. The syringe is now connected to the infusion line and the medication can be injected. Afterwards, the actuator body 2 can be pushed back again, the medication syringe removed and the fluid channel 21 can be cleaned using a newly prepared injection syringe with cleaning solution (e.g. 0.9% NaCl solution) into the drainage channel (remaining medication is expelled). Moving the actuator body 2 into the closed intermediate position brings the actuator fluid channel 21 into a closed position.

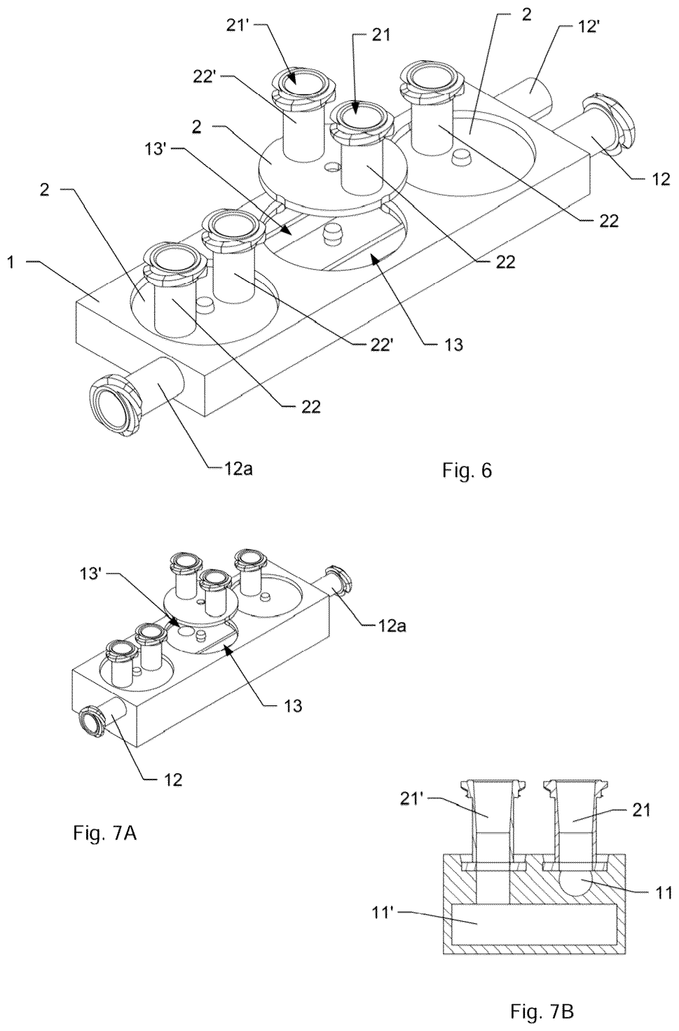

Several of the embodiments described above in FIGS. 1A-1D, 2A-2C, 3A-3B, 4A-4B, and 5A-C can be connected in sequence in an infusion line in order to increase the number of connection options for supply lines. Alternatively, the fluid control device can have a base body 1 with several actuator bodies 2. Such multi-fold systems are described in FIGS. 6, 7A-7B, 8A-8D, 9A-9D, 10A-10F, and 11A-11B.

In contrast to the embodiment in FIGS. 3A-3B, FIG. 6 shows a multi-fold system of the fluid control device with several disk-like, rotatable actuator bodies 2. These each have one or two separate actuator fluid channels 21, 21′, with actuator attachment nozzles 22, 22′ and actuator openings 23, 23′.

The multiple actuator bodies 2 are rotatably mounted next to each other in corresponding recesses in the base body 1. The base body 1 has a continuous base fluid channel 11 with a first base attachment nozzle 12 and an additional base attachment nozzle 12a. The continuous base fluid channel 11 has a base opening for each actuator body, via which the respective actuator fluid channels 21, 21′ can be connected.

The respective openings 13, 13′, 23, 23′ are designed in the same way as in the previously described embodiments with disk-like actuator body 2.

Furthermore, the base body 1 has a non-continuous base fluid channel 11′ which extends along the several control bodies 2 and which can also be connected to the actuator fluid channels 21, 21′ via base openings 13′.

Such a fluid control device can be used analogously to the preceding fluid control devices, with the difference that several connection options for supply lines are possible here.

The embodiment shown in FIGS. 5A-5C can also be constructed as a multi-fold system, in which several actuator bodies 2 are formed in a common base body 1.

FIGS. 7A-7B show a fluid control device similar to the embodiment in FIG. 6, but which, in contrast, has a chamber-like, non-continuous base fluid channel 11′. In FIG. 7A and FIG. 7B, the base attachment nozzle 12′, which can be located on the underside or on the side, is not visible. Such a base fluid channel 11′ serves as a drainage chamber (see FIG. 7B). The drainage chamber can contain a cassette, possibly with a suction element, which is replaced, or it can have a drain to periodically dispose of the drainage fluid or continuously drain it away.

Such a fluid control device can be used in the same way as the fluid control devices shown in FIGS. 1A-1D, 2A-2C, 3A-3B, 4A-4B, and 5A-C, with the difference that the multi-fold system allows several connection options for supply lines.

FIGS. 8A-8D show a further design of the fluid control device as a multi-fold system. In contrast to the variants already described with rotatable disks or a linearly displaceable plate, this variant has a circular cylinder-shaped actuator body 2 and a base body 1 with a corresponding recess. The sliding and sealing surface 20 of the actuator body 2 is formed by its lateral surface. The actuator body 2 has at least one actuator fluid channel 21, 21′ with an actuator attachment nozzle 22, 22′. In the embodiment shown, the at least one actuator attachment nozzle 22, 22′ runs parallel to the axis of rotation A. The at least one actuator fluid channel 21, 21′ describes a right angle and the corresponding actuator opening 23, 23′ is located in the lateral surface of the cylinder.

The actuator body 2 is mounted in the complementary recess of the base body 1 so that it can rotate about an axis of rotation A. The inner surface of the recess forms the sliding and sealing surface 10 of the base body 1.

The base openings 13, 13′ of the base fluid channels 11, 11′ are preferably arranged in such a way that the connection of an actuator fluid channel 21, 21′ between the two base fluid channels 11, 11′ can be changed with a 180° rotation of the actuator body 2. In the case of two actuator fluid channels 21, 21′, the actuator openings 23, 23′ of the actuator body are arranged symmetrically with 180° spacing around the axis of rotation A.

FIG. 8A shows a multi-fold system with three actuator bodies 2 connected in series. Similar to the fluid control devices in FIGS. 6 and 7A-7B, the base body 1 has two separate base fluid channels 11, 11′, each with a base attachment nozzle 12, 12′, one of which is continuous with an additional base attachment nozzle 12a. The base openings 13, 13′ are each located in the inner surface of the recess or the sliding and sealing surfaces 10 of the base body 1.

FIG. 8B shows a sectional view through the central actuator body 2 with only one actuator fluid channel 21. While one base opening 13′ is in line with the actuator opening 23′, the other base opening 13 is closed by the sliding and sealing surface 20 of the actuator body 2. In this embodiment of the fluid control device, the base fluid channels 11, 11′ are also separate from each other and cannot be connected to each other by the actuator body 2. In addition, the base fluid channels 11, 11′ of this fluid control device also have no branches or side arms within the base body 1. Even when the base opening is closed, the continuous base fluid channel 11 is completely flushed.

FIG. 8C shows a section of the base body 1 with the base opening 13 in the sliding and sealing surface 10 of the base body 1. FIG. 8D shows the actuator body 2 with the actuator opening 23 in the sliding and sealing surface 20 of the actuator body 2.

In this, as in the other embodiments, it is possible to provide both base fluid channels 11, 11′ as continuous base fluid channels with an additional base attachment nozzle 12a, 12a′. Such a fluid control device can be used analogously to the preceding fluid control devices from FIGS. 1A-1D, 2A-2C, 3A-3B, 4A-4B, and 5A-C, with the difference that several connection options for supply lines are possible here.

Such a device from FIGS. 8A-8D does not necessarily have to be designed as a multi-fold system, but can also be designed as a single fluid control device analogous to FIGS. 1A-1D, 2A-2C, 3A-3B, 4A-4B, and 5A-C.

An example of such a singular fluid control device with two actuator attachment nozzles 22, 22′ is shown in FIG. 16, although a variant with one actuator attachment nozzle is also possible. The fluid control device then has a circular cylindrical actuator body 2 and a sleeve-shaped base body 1. In contrast to the fluid control device shown in FIGS. 8A-8D, in the fluid control device shown in FIG. 16 the actuator attachment nozzles 22, 22′ of the actuator body 2 are not arranged parallel to the cylinder axis, but are inclined to the cylinder axis so that the free ends of the actuator attachment nozzles 22, 22′ are further apart in order to facilitate the connection of hoses. Such an inclined arrangement of the actuator attachment nozzles is also possible with other fluid control devices described. The base body 1 of the fluid control device in FIG. 16 also has a continuous base fluid channel 11, which ends in an additional base attachment nozzle 12a. One of the two base fluid channels 11 has two base attachment nozzles 12, 12a. However, the two base fluid channels 11, 11′ are also separate from each other and cannot be fluidically connected to each other via the actuator body. In the fluid control device shown in FIG. 16, the other base attachment nozzle 11′ is arranged in the radial direction of the base body 1, in contrast to a tangential arrangement in the fluid control device shown in FIG. 8A-8D.

Furthermore, the fluid control device in FIG. 16 can be designed without a continuous base fluid channel 11, i.e. similar to the fluid control devices in FIGS. 1A-1D and 2A-2C. In this case, both base attachment nozzles can be arranged in a radial direction.

FIGS. 9A-9D show a further embodiment of the fluid control device as a multi-fold system. In contrast to the variants already described, this variant has a circular cylinder-shaped base body 1 and several sleeve-shaped actuator bodies 2 rotating around the base body 1. The longitudinal axis of the cylindrical base body 1 forms an axis of rotation A around which the sleeve-shaped actuator body 2 can be rotated. The sliding and sealing surface 10 of the base body 1 is formed by its lateral surface. The sliding and sealing surface 20 of the actuator body 2 is formed by the inner surface of the sleeve.

In the embodiment shown, the multiple actuator bodies 1 have an actuator fluid channel 21 with an actuator attachment nozzle 22.

In the embodiment shown, the actuator attachment nozzle 22 runs perpendicular to the axis of rotation A and its actuator fluid channel 21 can be optionally connected to one of the base fluid channels 11, 11′ via the actuator opening 23.

As in the previous embodiments, the base openings 13, 13′ in the sliding and sealing surface 10 extend along the base fluid channels 11, 11′, so that these are open. In this way, no branches or side arms are necessary in the base fluid channels, which cannot be flushed or can only be flushed with difficulty when the base opening is closed.

A circumferential wall 19 of the base body 1 is arranged between the sleeve-shaped actuator bodies 2 to facilitate sealing. This can also have the latching means mentioned in the previous embodiments.

FIG. 9B shows a sectional view through the first actuator body 2 with only one actuator fluid channel 21. While one base opening 13 is in line with the actuator opening 23, the other base opening 13′ is closed by the sliding and sealing surface 20 of the actuator body 2. In this embodiment of the fluid control device, the base fluid channels 11, 11′ are also separate from each other and cannot be connected to each other by the actuator body 2. In addition, the base fluid channels 11, 11′ of this fluid control device also have no branches or side arms within the base body 1. Even when the base opening is closed, the continuous base fluid channel 11 is completely flushed.

FIG. 9C shows the base body 1 with the base apertures 13, 13′ in the sliding and sealing surface 10 of the base body 1. FIG. 9D shows the actuator body 2 with the actuator opening 23 in the sliding and sealing surface 20 of the actuator body 2, which forms the inner surface of the actuator body 2.

The base fluid channels 11, 11′ can also be arranged symmetrically at a distance of 180° around the axis of rotation A, so that the actuator body 2 can have two diametrically opposed actuator fluid channels 21, 21′ or actuator openings 23, 23′, which allow a quick change as with the so-called quick exchange.

The fluid control device in FIGS. 9A-9D can also be realized as a simple variant with only one actuator body.

Such a fluid control device can be used analogously to the preceding fluid control devices in FIGS. 1A-1D, 2A-2C, 3A-3B, 4A-4B, and 5A-C, with the difference that the multi-fold system allows several connection options for supply lines.

FIGS. 10A-10F show a further embodiment of the fluid control device as a multi-fold system. In this embodiment, the base body 1 is cylindrical in shape and has several circular cylindrical actuator bodies 2 on its cylindrical circumferential surface, which are sealingly and rotatably embedded in corresponding recesses in the base body 1.

The base body 1 has two separate base fluid channels 11, 11′, which are spaced apart from each other in the axial direction and have a central circular chamber 11a, 11a′. A first base fluid channel 11 has two base attachment nozzles 12, 12a, which are arranged laterally on the lateral surface of the cylindrical base body 1. The second base fluid channel 11′, which can be used as drainage, has a base attachment nozzle 12′, which is arranged on an end face of the cylindrical base body 1 and parallel to a central axis of the cylindrical base body 1.

The multiple circular-cylindrical actuator bodies 1 have one or two separate actuator fluid channels 21, 21′ with respective actuator attachment nozzles 22, 22′ and actuator openings 23, 23′. By rotating the control elements 2, their actuator openings 23, 23′ can be brought into alignment with the base openings 13, 13′ of the central chambers 11a, 11a′ of the base fluid channels 13, 13′.

FIG. 10B shows a sectional view transverse to the central axis and through the first base fluid channel 11. FIG. 10D shows a sectional view transverse to the central axis and through the second base fluid channel 11′. FIG. 10C shows along the center axis and through both base fluid channels 11, 11′.

FIG. 10E shows a side view of the base body 1, in which the sliding and sealing surface 10 of the base body 1 and the base openings 13, 13′ are visible. FIG. 10F shows a perspective view of the actuator body 2, in which the sliding and sealing surface 20 of the actuator body 2 and the actuator openings 23, 23′ are visible.

In the fluid control device shown in FIGS. 10A-10F, the actuator fluid channels 21, 21′ run in a radial direction to the central axis of the base body 1 and can be connected to the central chambers 11a, 11a′ of the base fluid channels. The central chamber 11a of the first base fluid channel 11 with the two base attachment nozzles 12, 12a can, for example, be flushed by an infusion. Depending on the position of the actuator bodies 2, syringe pumps or ordinary syringes, for example, can be connected to the infusion. The central chamber 11a′ of the second base fluid channel 11′ serves as a collecting basin for the drainage.

Alternatively, the actuator body 2 can also be tapered so that it tapers towards the sliding and sealing surface 20. In this way, even more actuator bodies 2 can be accommodated in the base body 1 and/or the central chambers 11a, 11a′ can be made smaller.

FIGS. 17A-17B show a further embodiment of the fluid control device as a multi-fold system—similar to the fluid control device in FIGS. 10A-10F. In the fluid control device in FIGS. 17A-17B, the base body 1 has several sleeve-shaped sections arranged around a central axis, in each of which a circular cylinder-shaped actuator body 2 is accommodated. The actuator bodies 2 can be designed similarly to those already described and have one or two actuator attachment nozzles 22, 22′. The base body 1 has two separate base fluid channels 11, 11′, each of which forms a central chamber 11a, 11a′, which can be fluidically connected to the respective actuator fluid channels 21, 21′ of the control elements 2—depending on the position of the control element 2. The base attachment nozzles 12, 12a of the continuous first base channel 11 are arranged at an angle of 30 to 60 degrees to the central axis. The base attachment nozzle 12′ of the second base channel 11 is arranged parallel to the central axis.

FIG. 17B shows a side view of the base body 1, in which the sliding and sealing surface 10 of the base body 1 and the base openings 13, 13′ can be seen. The first base openings 13 to the continuous first base channel 11 or the chamber of the continuous base channel 11 are larger in this embodiment so that no undercuts are formed towards the chamber, which can lead to problems during injection molding. The chamber of the second base channel 11′, which is usually used as a drain, connects its second base openings with the second base attachment nozzle 12′. Undercuts are also avoided here.

FIGS. 11A-11B show a further embodiment of the fluid control device, which differs from the fluid control device in FIGS. 10A-10F in that the second base fluid channel is designed analogously to the first base fluid channel 11 with a central chamber 11a′ and has a base attachment nozzle 12′ arranged laterally on the lateral surface. FIG. 11B shows a sectional view along the central axis and through the base fluid channels 11, 11′.

The fluid control devices in FIGS. 10A-10F and FIGS. 11A-11B can be used in the same way as the preceding fluid control devices in FIGS. 1A-1D, 2A-2C, 3A-3B, 4A-4B, and 5A-C, whereby several connection options for supply lines are possible with the multi-fold system.

FIGS. 12A-12D, 13, and 14 show various embodiments of a fluid control device in which the actuator body 2 has only one actuator fluid channel 21 with actuator attachment nozzle 22. In contrast to the preceding embodiments with two base fluid channels, however, the base body 1 has more than two base fluid channels 11 with base attachment nozzles 12. The actuator fluid channel 21 can each be connected to one of the several base fluid channels 11.

Such fluid control devices allow, for example, the aspiration of fluids from the catheter system, which is connected via one of the connecting ports of the base body, and the discharge of the aspirate including air into the drainage channel, which is connected to another connecting port of the base body. A liquid medium can also be aspirated from one of the other connecting pieces (supply ports) and injected into the catheter system. Residual liquids and air in the syringe can be disposed of via the drainage system.

FIGS. 12A-12D show a variant in which the actuator body 2—similar to the embodiment in FIGS. 8A-8D—is designed as a rotatable circular cylinder, the outer surface of which forms the sliding and sealing surface 20 with the actuator opening 23. The base body 2 is correspondingly designed as a sleeve or hollow cylinder and has the complementary sliding and sealing surface 10 with the respective base openings on the inside. Conversely, it is also possible to form the actuator body with an actuator attachment nozzle as a sleeve or hollow cylinder, in which case the base body with the multiple base attachment nozzles forms the inner circular cylinder (similar to the embodiment in FIGS. 9A-9D).

FIG. 13 shows a variant in which the actuator body 2—similar to the embodiment in FIGS. 5A-5C—is designed as a linearly displaceable plate, which is accommodated in the base body 1 in a linearly displaceable manner. The actuator attachment nozzle 22 is arranged on the upper side (i.e. the side facing away from the base body). In the embodiment shown, the base attachment nozzles are arranged laterally. Accordingly, the bend of the fluid channel can be arranged in the actuator fluid channel 21 of the actuator body 2 or in the base fluid channel 11 of the base body 1. In the first case, the plate of the control element 1 is thicker and the sliding and sealing surface with the actuator opening is arranged at the side. In the second case, the sliding and sealing surface is arranged on the underside of the plate. Alternatively, the base attachment nozzles can be arranged on the underside of the base body, i.e. the side facing away from the actuator body, so that there is no bend in the fluid channels.

FIG. 14 shows a variant similar to the embodiment in FIGS. 1A-1D with two circular disks that can rotate in relation to each other, whereby the base attachment nozzles are arranged at regular intervals around the axis of rotation of the actuator body.

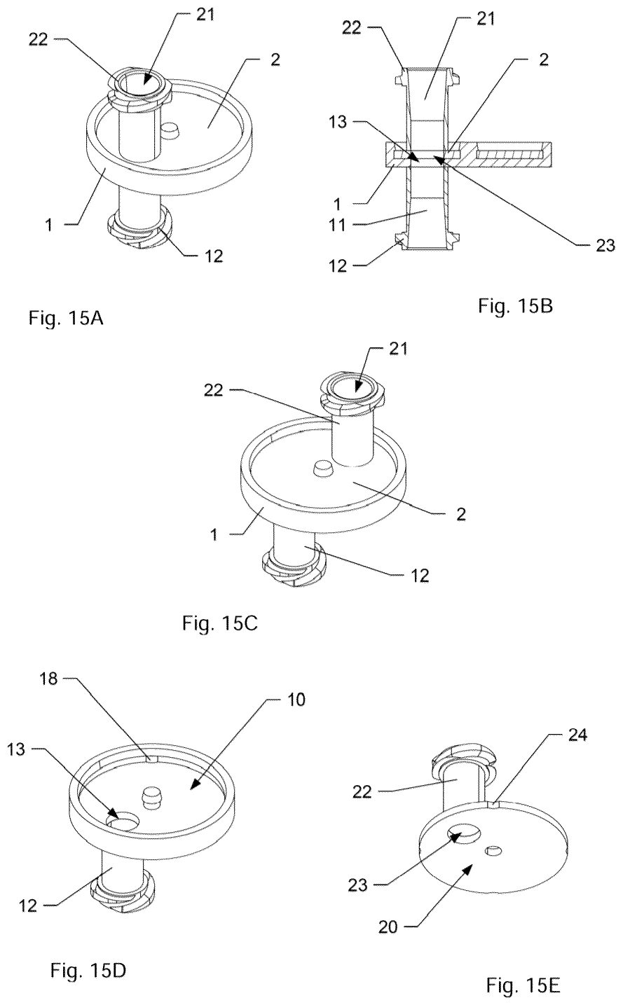

Finally, FIGS. 15A-15E show an infusion stopper based on the simplest embodiment of a medical fluid control device for changing and controlling fluid flows. FIG. 15A and FIG. 15C each show a perspective view. FIG. 15B shows a sectional view. The fluid control device comprises a base body 1 (FIG. 15D) and an actuator body 2 (FIG. 15E), wherein the base body 1 and the actuator 2 each have a sliding and sealing surface 10, 20 facing each other and the actuator body 2 is held in the base body 1 in a sealing and movable manner. In the embodiment shown, the actuator is designed as a rotatable disk with a circular sliding and sealing surface. Other variants, e.g. a linearly displaceable plate, a rotatable cylinder (sliding and sealing surface on the outer surface) or a rotatable sleeve (sliding and sealing surface on the inner surface) are also possible and are described in connection with other embodiments in the other figures. The sliding and sealing surface of the base body is correspondingly complementary to the sliding and sealing surface of the actuator body.

In the infusion stopper, the actuator body 1 only has an actuator fluid channel 21 with an actuator attachment nozzle 22. The actuator fluid channel 21 ends in an actuator opening 23 in the sliding and sealing surface 20 of the actuator body 2. The base body 1 also only has a base attachment nozzle 11 with a base fluid channel 11, the base opening 13 of which is located in the sliding and sealing surface 10 of the base body 1. In an open position (FIG. 15A) of the infusion body, the two openings 13, 23 are in line with each other. In a closed position (FIG. 15C), the openings 13, 23 each lie directly on the sliding and sealing surface of the counterpart. There is no dead volume or diameter reduction due to a central rotating element in the lumen, as is the case with a conventional stopcock, for example.

The infusion stopper allows the regulation (go/stop) of a fluid flow without increasing the flow resistance in the open state, as the diameters of the fluid channels are the same. There is no intermediate channel with a smaller diameter, as is also known from conventional stopcocks. In addition, the infusion stopper makes it easy to see whether the line is closed or open. The infusion stopper can be combined with other fluid control devices in infusion systems and installed upstream and/or downstream. The infusion stopper thus makes it possible to influence the direction of flow of an aspiration or injection into the injection channel.

The connecting pieces of all embodiments can have a so-called Luer connection. The base attachment nozzles 12 shown on the base body 1 of all embodiments, which have a so-called Luer connection, are used for aspiration or infusion. Only one base attachment nozzle may not have a Luer connection. This is generally used as a drainage channel or waste channel and is easily recognizable in this way. The positioning attachment nozzles shown also have a Luer connection.

FIG. 18 shows an example of a fluid control device with a first pressure measuring device 30 on the first base fluid channel 11 and a second pressure measuring device on the second base fluid channel 11′.

All embodiments can be regarded as independent inventions. Individual features, even if described in connection with specific embodiments, can also be combined with other embodiments.

### REFERENCE SIGNS

1 base body
10 sliding and sealing surface
11, 11′ fluid channel of base body (base fluid channel)
11a, 11a′ central chamber
12, 12′ attachment nozzle of the base body (base attachment nozzle)
12a additional base attachment nozzle
13, 13′ opening in the sliding and sealing surface of the base body (base opening)
14 surrounding wall
15 projection
16 pin
17 latching means
18 latching lug
19 surrounding wall
2, 2′, 2″ actuator body
20 sliding and sealing surface of the actuator body
21, 21′ fluid channel of the actuator body (actuator fluid channel)
22, 22′ attachment nozzle of the actuator body (actuator attachment nozzle)
23, 23′ opening in the sliding and sealing surface of the actuator body (actuator opening)
24 latching notch
30 pressure measuring device

1-16. (canceled)

17. A medical fluid control device for changing and controlling flows of fluid, the device comprising:
a base body and an actuator body which is movable relative to the base body;
wherein the base body and the actuator body have, respectively, a sliding and sealing surface facing each other and the actuator body is fastened movably to the base body;
wherein the actuator body has at least one actuator fluid channel, at least one actuator attachment nozzle for attaching hoses or syringes, and at least one actuator opening in the sliding and sealing surface of the actuator body, and wherein each actuator fluid channel in each case connects an actuator attachment nozzle directly to an actuator opening in the sliding and sealing surface of the actuator body;
wherein the base body has a first base fluid channel and a second base fluid channel with in each case a base attachment nozzle, wherein the first base fluid channel and the second base fluid channel are independent of each other and are not directly fluidically connectable to each other via the actuator body;
wherein the first base fluid channel and the second base fluid channel each comprise a base opening in the sliding and sealing surface of the base body and are free of a branching in the base body; and
wherein the actuator body is movable in such a way that the at least one actuator fluid channel can selectably be brought into a closed position or can be brought into an open position in connection with the first or the second base fluid channel.

18. The medical fluid control device according to claim 17, wherein the actuator body is rotatable through 360° about an axis of rotation and has a rotationally symmetrical sliding and sealing surface.

19. The medical fluid control device according to claim 17, wherein the first and second base openings are arranged in the sliding and sealing surface at a distance of 180° from one another with the same radius relative to the axis of rotation, so that the at least one actuator opening of the actuator body can be moved from the first to the second base opening with a 180° rotation.

20. The medical fluid control device according to claim 17, wherein the actuator body is in the form of a rotatable disc with a circular sliding and sealing surface, in the form of a circular sleeve with the sliding and sealing surface on the inner side, in the form of a circular cylinder with the sliding and sealing surface on the circumferential surface or in the form of a circular cylinder with the sliding and sealing surface on the end face, and the sliding and sealing surface of the base body is in each case complementary to the sliding and sealing surface of the actuator body.

21. The medical fluid control device according to claim 17, wherein the actuator body is designed in the form of a linearly displaceable plate with the sliding and sealing surface, and the sliding and sealing surface of the base body is designed to be complementary to the sliding and sealing surface of the actuator body.

22. The medical fluid control device according to claim 17, wherein the fluid control device has a plurality of actuators bodies which are movable relative to the base body and which are movably fastened in a plurality of recesses on the base body.

23. The medical fluid control device according to claim 17, wherein the first base fluid channel and/or the second base fluid channel has a second, additional base attachment nozzle, so that at least one base fluid channel forms a continuous base fluid channel in the base body with base attachment nozzles on both sides, irrespective of the position of the actuator body.

24. The medical fluid control device according to claim 23, wherein the continuous base fluid channel is guided so as to break through the sliding and sealing surface of the base body and thereby forms the base opening of the base fluid channel.

25. The medical fluid control device according to claim 17, wherein the base fluid channel has no dead leg irrespective of the position of the actuator.

26. The medical fluid control device according to claim 17, wherein the base fluid channel is open in the region of the sliding and sealing surface of the base body, so that the base opening of the base fluid channel extends along the sliding and sealing surface of the base body.

27. The medical fluid control device according to claim 17, wherein the actuator body has a single actuator fluid channel and the base body has more than two base fluid channels.

28. The medical fluid control device according to claim 17, wherein the base body and the actuator body each have complementary latching means, so that the actuator body can be latched in different positions.

29. The medical fluid control device according to claim 17, wherein at least one base attachment nozzle and the at least one actuator attachment nozzle is designed as a Luer connection.

30. The medical fluid control device according to claim 17, wherein the sliding and sealing surfaces of the actuator body and the base body are in sealing contact with one another.

31. The medical fluid control device according to claim 17, wherein the fluid control device is manufactured as a disposable article made of plastic.

32. The medical fluid control device according to claim 17, wherein the first base fluid channel and/or the second base fluid channel is designed with a pressure measuring device.
